SEQUENCE LISTING (150 sequences)

SEQ ID NO: 1 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 2 - DNA, 798 bp, Homo sapiens
  source 1..798: mol_type = genomic DNA
atgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatga

SEQ ID NO: 3 - protein, 240 aa, Homo sapiens
QVQLVQSGAEVKKPGASVKVSCKASGYTFTSYAMDWVRQAPGQGLEWMGAISPSGGGTVYNEKFQGRVTMTRDTSTSTVYMELSSLRSEDTAVYYCAREYWGELFDLWGQGTLVTVSSGGGGSGGGGSGGGGSDIQMTQSPSSLSASVGDRVTITCRASQDIGNWLNWYQQKPGKAPKLLIYGASNLQSGVPSRFSGSGSGTDFTLTISSLQPEDFATYYCQQAYGYPFTFGQGTKVEIK

SEQ ID NO: 4 - protein, 240 aa, Homo sapiens
QVQLVQSGAEVKKPGASVKVSCKASGYTFTSYSMSWVRQAPGQGLEWMGVISSGTGSTYYNEKFQGRVTMTRDTSTSTVYMELSSLRSEDTAVYYCAKNSWGDDFDLWGQGTLVTVSSGGGGSGGGGSGGGGSDIQMTQSPSSLSASVGDRVTITCQASQNINNFLAWYQQKPGKAPKLLIYDASNLATGVPSRFSGSGSGTDFTLTISSLQPEDFATYYCQQSYNTPLTFGQGTKVEIK

SEQ ID NO: 5 - protein, 240 aa, Homo sapiens
QVQLVQSGAEVKKPGASVKVSCKASGYTFTSYDIDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIK

SEQ ID NO: 6 - protein, 240 aa, Homo sapiens
QVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSSGGGGSGGGGSGGGGSDIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIK

SEQ ID NO: 7 - protein, 240 aa, Homo sapiens
QVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSSGGGGSGGGGSGGGGSDIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIK

SEQ ID NO: 8 - protein, 240 aa, Homo sapiens
QVQLVQSGAEVKKPGASVKVSCKASGYTFTDYGMDWVRQAPGQGLEWMGYISGGTGRTAYNEKFQGRVTMTRDTSTSTVYMELSSLRSEDTAVYYCARYGANDGFDIWGQGTLVTVSSGGGGSGGGGSGGGGSDIQMTQSPSSLSASVGDRVTITCRASQDISSYLNWYQQKPGKAPKLLIYGASRLYTGVPSRFSGSGSGTDFTLTISSLQPEDFATYYCQQGYNTPYTFGGGTKVEIK

SEQ ID NO: 9 - protein, 240 aa, Homo sapiens
QVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIK

SEQ ID NO: 10 - protein, 240 aa, Homo sapiens
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARPDHGDFMDVWGQGTLVTVSSGGGGSGGGGSGGGGSDIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIK

SEQ ID NO: 11 - protein, 241 aa, Homo sapiens
QVQLVQSGAEVKKPGASVKVSCKASGYTFTDYDMSWVRQAPGQGLEWMGVISSSGGSTTYNEKFQGRVTMTRDTSTSTVYMELSSLRSEDTAVYYCARSPMNEDWFDYWGQGTLVTVSSGGGGSGGGGSGGGGSDIQMTQSPSSLSASVGDRVTITCKASQGVSTWLNWYQQKPGKAPKLLIYVASRLESGVPSRFSGSGSGTDFTLTISSLQPEDFATYYCQQSSFLPITFGGGTKVEIK

SEQ ID NO: 12 - protein, 240 aa, Homo sapiens
QVQLVQSGAEVKKPGASVKVSCKASGYTFTHYAMSWVRQAPGQGLEWMGGIYGGGDSTNYNEKFQGRVTMTRDTSTSTVYMELSSLRSEDTAVYYCAKASWEDTFDYWGQGTLVTVSSGGGGSGGGGSGGGGSDIQMTQSPSSLSASVGDRVTITCKASQDIRNYLAWYQQKPGKAPKLLIYGASSLASGVPSRFSGSGSGTDFTLTISSLQPEDFATYYCQQSYNYPLTFGQGTKVEIK

SEQ ID NO: 13 - protein, 118 aa, Homo sapiens
QVQLVQSGAEVKKPGASVKVSCKASGYTFTSYAMDWVRQAPGQGLEWMGAISPSGGGTVYNEKFQGRVTMTRDTSTSTVYMELSSLRSEDTAVYYCAREYWGELFDLWGQGTLVTVSS

SEQ ID NO: 14 - protein, 118 aa, Homo sapiens
QVQLVQSGAEVKKPGASVKVSCKASGYTFTSYSMSWVRQAPGQGLEWMGVISSGTGSTYYNEKFQGRVTMTRDTSTSTVYMELSSLRSEDTAVYYCAKNSWGDDFDLWGQGTLVTVSS

SEQ ID NO: 15 - protein, 118 aa, Homo sapiens
QVQLVQSGAEVKKPGASVKVSCKASGYTFTSYDIDWVRQAPGQGLEWMGGISPQSGGTSYNEKFQGRVTMTRDTSTSTVYMELSSLRSEDTAVYYCARVHGDYHFDYWGQGTLVTVSS

SEQ ID NO: 16 - protein, 118 aa, Homo sapiens
QVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSS

SEQ ID NO: 17 - protein, 118 aa, Homo sapiens
QVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSS

SEQ ID NO: 18 - protein, 118 aa, Homo sapiens
QVQLVQSGAEVKKPGASVKVSCKASGYTFTDYGMDWVRQAPGQGLEWMGYISGGTGRTAYNEKFQGRVTMTRDTSTSTVYMELSSLRSEDTAVYYCARYGANDGFDIWGQGTLVTVSS

SEQ ID NO: 19 - protein, 118 aa, Homo sapiens
QVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSS

SEQ ID NO: 20 - protein, 118 aa, Homo sapiens
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARPDHGDFMDVWGQGTLVTVSS

SEQ ID NO: 21 - protein, 119 aa, Homo sapiens
QVQLVQSGAEVKKPGASVKVSCKASGYTFTDYDMSWVRQAPGQGLEWMGVISSSGGSTTYNEKFQGRVTMTRDTSTSTVYMELSSLRSEDTAVYYCARSPMNEDWFDYWGQGTLVTVSS

SEQ ID NO: 22 - protein, 118 aa, Homo sapiens
QVQLVQSGAEVKKPGASVKVSCKASGYTFTHYAMSWVRQAPGQGLEWMGGIYGGGDSTNYNEKFQGRVTMTRDTSTSTVYMELSSLRSEDTAVYYCAKASWEDTFDYWGQGTLVTVSS

SEQ ID NO: 23 - protein, 5 aa, Homo sapiens
SYAMD

SEQ ID NO: 24 - protein, 5 aa, Homo sapiens
SYSMS

SEQ ID NO: 25 - protein, 5 aa, Homo sapiens
SYDID

SEQ ID NO: 26 - protein, 5 aa, Homo sapiens
SYDMS

SEQ ID NO: 27 - protein, 5 aa, Homo sapiens
DYAMD

SEQ ID NO: 28 - protein, 5 aa, Homo sapiens
DYGMD

SEQ ID NO: 29 - protein, 5 aa, Homo sapiens
HYAMD

SEQ ID NO: 30 - protein, 5 aa, Homo sapiens
SYAMS

SEQ ID NO: 31 - protein, 5 aa, Homo sapiens
DYDMS

SEQ ID NO: 32 - protein, 5 aa, Homo sapiens
HYAMS

SEQ ID NO: 33 - protein, 17 aa, Homo sapiens
AISPSGGGTVYNEKFQG

SEQ ID NO: 34 - protein, 17 aa, Homo sapiens
VISSGTGSTYYNEKFQG

SEQ ID NO: 35 - protein, 17 aa, Homo sapiens
GISPQSGGTSYNEKFQG

SEQ ID NO: 36 - protein, 17 aa, Homo sapiens
AINPSGDFTSYNEKFQG

SEQ ID NO: 37 - protein, 17 aa, Homo sapiens
WISGQSGSTSYNEKFQG

SEQ ID NO: 38 - protein, 17 aa, Homo sapiens
YISGGTGRTAYNEKFQG

SEQ ID NO: 39 - protein, 17 aa, Homo sapiens
GISPQSGGTSYNEKFQG

SEQ ID NO: 40 - protein, 17 aa, Homo sapiens
AINSQGKSTYYADSVKG

SEQ ID NO: 41 - protein, 17 aa, Homo sapiens
VISSSGGSTTYNEKFQG

SEQ ID NO: 42 - protein, 17 aa, Homo sapiens
GIYGGGDSTNYNEKFQG

SEQ ID NO: 43 - protein, 10 aa, Homo sapiens
REYWGELFDL

SEQ ID NO: 44 - protein, 10 aa, Homo sapiens
KNSWGDDFDL

SEQ ID NO: 45 - protein, 10 aa, Homo sapiens
RVHGDYHFDY

SEQ ID NO: 46 - protein, 9 aa, Homo sapiens
GVGWSFLDY

SEQ ID NO: 47 - protein, 10 aa, Homo sapiens
KWLDGPWFDV

SEQ ID NO: 48 - protein, 10 aa, Homo sapiens
RYGANDGFDI

SEQ ID NO: 49 - protein, 10 aa, Homo sapiens
RVHGDYHFDY

SEQ ID NO: 50 - protein, 10 aa, Homo sapiens
RPDHGDFMDV

SEQ ID NO: 51 - protein, 11 aa, Homo sapiens
RSPMNEDWFDY

SEQ ID NO: 52 - protein, 10 aa, Homo sapiens
KASWEDTFDY

SEQ ID NO: 53 - protein, 107 aa, Homo sapiens
DIQMTQSPSSLSASVGDRVTITCRASQDIGNWLNWYQQKPGKAPKLLIYGASNLQSGVPSRFSGSGSGTDFTLTISSLQPEDFATYYCQQAYGYPFTFGQGTKVEIK

SEQ ID NO: 54 - protein, 107 aa, Homo sapiens
DIQMTQSPSSLSASVGDRVTITCQASQNINNFLAWYQQKPGKAPKLLIYDASNLATGVPSRFSGSGSGTDFTLTISSLQPEDFATYYCQQSYNTPLTFGQGTKVEIK

SEQ ID NO: 55 - protein, 107 aa, Homo sapiens
DIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIK

SEQ ID NO: 56 - protein, 107 aa, Homo sapiens
DIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIK

SEQ ID NO: 57 - protein, 107 aa, Homo sapiens
DIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIK

SEQ ID NO: 58 - protein, 107 aa, Homo sapiens
DIQMTQSPSSLSASVGDRVTITCRASQDISSYLNWYQQKPGKAPKLLIYGASRLYTGVPSRFSGSGSGTDFTLTISSLQPEDFATYYCQQGYNTPYTFGGGTKVEIK

SEQ ID NO: 59 - protein, 107 aa, Homo sapiens; identical to SEQ ID NO: 55

SEQ ID NO: 60 - protein, 107 aa, Homo sapiens
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIK

SEQ ID NO: 61 - protein, 107 aa, Homo sapiens
DIQMTQSPSSLSASVGDRVTITCKASQGVSTWLNWYQQKPGKAPKLLIYVASRLESGVPSRFSGSGSGTDFTLTISSLQPEDFATYYCQQSSFLPITFGGGTKVEIK

SEQ ID NO: 62 - protein, 107 aa, Homo sapiens
DIQMTQSPSSLSASVGDRVTITCKASQDIRNYLAWYQQKPGKAPKLLIYGASSLASGVPSRFSGSGSGTDFTLTISSLQPEDFATYYCQQSYNYPLTFGQGTKVEIK

SEQ ID NO: 63 - protein, 11 aa, Homo sapiens
RASQDIGNWLN

SEQ ID NO: 64 - protein, 11 aa, Homo sapiens
QASQNINNFLA

SEQ ID NO: 65 - protein, 11 aa, Homo sapiens
QASQDVSTDLA

SEQ ID NO: 66 - protein, 11 aa, Homo sapiens
QASQDVRTWLA

SEQ ID NO: 67 - protein, 11 aa, Homo sapiens
KASEDIHSDLA

SEQ ID NO: 68 - protein, 11 aa, Homo sapiens
RASQDISSYLN

SEQ ID NO: 69 - protein, 11 aa, Homo sapiens
QASQDVSTDLA

SEQ ID NO: 70 - protein, 11 aa, Homo sapiens
RASQGISNWLA

SEQ ID NO: 71 - protein, 11 aa, Homo sapiens
KASQGVSTWLN

SEQ ID NO: 72 - protein, 11 aa, Homo sapiens
KASQDIRNYLA

SEQ ID NO: 73 - protein, 7 aa, Homo sapiens
GASNLQS

SEQ ID NO: 74 - protein, 7 aa, Homo sapiens
DASNLAT

SEQ ID NO: 75 - protein, 7 aa, Homo sapiens
HASSLES

SEQ ID NO: 76 - protein, 7 aa, Homo sapiens
SASSLQT

SEQ ID NO: 77 - protein, 7 aa, Homo sapiens
SASSLQS

SEQ ID NO: 78 - protein, 7 aa, Homo sapiens
GASRLYT

SEQ ID NO: 79 - protein, 7 aa, Homo sapiens
HASSLES

SEQ ID NO: 80 - protein, 7 aa, Homo sapiens
GASSLQS

SEQ ID NO: 81 - protein, 7 aa, Homo sapiens
VASRLES

SEQ ID NO: 82 - protein, 7 aa, Homo sapiens
GASSLAS

SEQ ID NO: 83 - protein, 9 aa, Homo sapiens
QQAYGYPFT

SEQ ID NO: 84 - protein, 9 aa, Homo sapiens
QQSYNTPLT

SEQ ID NO: 85 - protein, 9 aa, Homo sapiens
QQYYNYPLT

SEQ ID NO: 86 - protein, 9 aa, Homo sapiens
QQSNYLPFT

SEQ ID NO: 87 - protein, 9 aa, Homo sapiens
QQAYYLPLT

SEQ ID NO: 88 - protein, 9 aa, Homo sapiens
QQGYNTPYT

SEQ ID NO: 89 - protein, 9 aa, Homo sapiens
QQYYNYPLT

SEQ ID NO: 90 - protein, 9 aa, Homo sapiens
QQYSSFPTT

SEQ ID NO: 91 - protein, 9 aa, Homo sapiens
QQSSFLPIT

SEQ ID NO: 92 - protein, 9 aa, Homo sapiens
QQSYNYPLT

SEQ ID NO: 93 - protein, 489 aa, Homo sapiens
MALPVTALLLPLALLLHAARPQVQLVQSGAEVKKPGASVKVSCKASGYTFTSYAMDWVRQAPGQGLEWMGAISPSGGGTVYNEKFQGRVTMTRDTSTSTVYMELSSLRSEDTAVYYCAREYWGELFDLWGQGTLVTVSSGGGGSGGGGSGGGGSDIQMTQSPSSLSASVGDRVTITCRASQDIGNWLNWYQQKPGKAPKLLIYGASNLQSGVPSRFSGSGSGTDFTLTISSLQPEDFATYYCQQAYGYPFTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 94 - protein, 489 aa, Homo sapiens
MALPVTALLLPLALLLHAARPQVQLVQSGAEVKKPGASVKVSCKASGYTFTSYSMSWVRQAPGQGLEWMGVISSGTGSTYYNEKFQGRVTMTRDTSTSTVYMELSSLRSEDTAVYYCAKNSWGDDFDLWGQGTLVTVSSGGGGSGGGGSGGGGSDIQMTQSPSSLSASVGDRVTITCQASQNINNFLAWYQQKPGKAPKLLIYDASNLATGVPSRFSGSGSGTDFTLTISSLQPEDFATYYCQQSYNTPLTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 95 - protein, 489 aa, Homo sapiens
MALPVTALLLPLALLLHAARPQVQLVQSGAEVKKPGASVKVSCKASGYTFTSYDIDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 96 - protein, 489 aa, Homo sapiens
MALPVTALLLPLALLLHAARPQVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSSGGGGSGGGGSGGGGSDIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - protein, 489 aa, Homo sapiens
MALPVTALLLPLALLLHAARPQVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSSGGGGSGGGGSGGGGSDIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 98 - protein, 489 aa, Homo sapiens
MALPVTALLLPLALLLHAARPQVQLVQSGAEVKKPGASVKVSCKASGYTFTDYGMDWVRQAPGQGLEWMGYISGGTGRTAYNEKFQGRVTMTRDTSTSTVYMELSSLRSEDTAVYYCARYGANDGFDIWGQGTLVTVSSGGGGSGGGGSGGGGSDIQMTQSPSSLSASVGDRVTITCRASQDISSYLNWYQQKPGKAPKLLIYGASRLYTGVPSRFSGSGSGTDFTLTISSLQPEDFATYYCQQGYNTPYTFGGGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 99 - protein, 489 aa, Homo sapiens
MALPVTALLLPLALLLHAARPQVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 100 - protein, 489 aa, Homo sapiens
MALPVTALLLPLALLLHAARPEVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARPDHGDFMDVWGQGTLVTVSSGGGGSGGGGSGGGGSDIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 101 - protein, 490 aa, Homo sapiens
MALPVTALLLPLALLLHAARPQVQLVQSGAEVKKPGASVKVSCKASGYTFTDYDMSWVRQAPGQGLEWMGVISSSGGSTTYNEKFQGRVTMTRDTSTSTVYMELSSLRSEDTAVYYCARSPMNEDWFDYWGQGTLVTVSSGGGGSGGGGSGGGGSDIQMTQSPSSLSASVGDRVTITCKASQGVSTWLNWYQQKPGKAPKLLIYVASRLESGVPSRFSGSGSGTDFTLTISSLQPEDFATYYCQQSSFLPITFGGGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 102 - protein, 489 aa, Homo sapiens
MALPVTALLLPLALLLHAARPQVQLVQSGAEVKKPGASVKVSCKASGYTFTHYAMSWVRQAPGQGLEWMGGIYGGGDSTNYNEKFQGRVTMTRDTSTSTVYMELSSLRSEDTAVYYCAKASWEDTFDYWGQGTLVTVSSGGGGSGGGGSGGGGSDIQMTQSPSSLSASVGDRVTITCKASQDIRNYLAWYQQKPGKAPKLLIYGASSLASGVPSRFSGSGSGTDFTLTISSLQPEDFATYYCQQSYNYPLTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 103 - protein, 472 aa, Homo sapiens
MALPVTALLLPLALLLHAARPQVQLVQSGAEVKKPGASVKVSCKASGYTFTSYAMDWVRQAPGQGLEWMGAISPSGGGTVYNEKFQGRVTMTRDTSTSTVYMELSSLRSEDTAVYYCAREYWGELFDLWGQGTLVTVSSGGGGSGGGGSGGGGSDIQMTQSPSSLSASVGDRVTITCRASQDIGNWLNWYQQKPGKAPKLLIYGASNLQSGVPSRFSGSGSGTDFTLTISSLQPEDFATYYCQQAYGYPFTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 104 - protein, 472 aa, Homo sapiens
MALPVTALLLPLALLLHAARPQVQLVQSGAEVKKPGASVKVSCKASGYTFTSYSMSWVRQAPGQGLEWMGVISSGTGSTYYNEKFQGRVTMTRDTSTSTVYMELSSLRSEDTAVYYCAKNSWGDDFDLWGQGTLVTVSSGGGGSGGGGSGGGGSDIQMTQSPSSLSASVGDRVTITCQASQNINNFLAWYQQKPGKAPKLLIYDASNLATGVPSRFSGSGSGTDFTLTISSLQPEDFATYYCQQSYNTPLTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 105 - protein, 472 aa, Homo sapiens
MALPVTALLLPLALLLHAARPQVQLVQSGAEVKKPGASVKVSCKASGYTFTSYDIDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 106 - protein, 472 aa, Homo sapiens
MALPVTALLLPLALLLHAARPQVQLVQSGAEVKKPGASVKVSCKASGYTFTSYDMSWVRQAPGQGLEWMGAINPSGDFTSYNEKFQGRVTMTRDTSTSTVYMELSSLRSEDTAVYYCARGVGWSFLDYWGQGTLVTVSSGGGGSGGGGSGGGGSDIQMTQSPSSLSASVGDRVTITCQASQDVRTWLAWYQQKPGKAPKLLIYSASSLQTGVPSRFSGSGSGTDFTLTISSLQPEDFATYYCQQSNYLPFTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 107 - protein, 472 aa, Homo sapiens
MALPVTALLLPLALLLHAARPQVQLVQSGAEVKKPGASVKVSCKASGYTFTDYAMDWVRQAPGQGLEWMGWISGQSGSTSYNEKFQGRVTMTRDTSTSTVYMELSSLRSEDTAVYYCAKWLDGPWFDVWGQGTLVTVSSGGGGSGGGGSGGGGSDIQMTQSPSSLSASVGDRVTITCKASEDIHSDLAWYQQKPGKAPKLLIYSASSLQSGVPSRFSGSGSGTDFTLTISSLQPEDFATYYCQQAYYLPLTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 108 - protein, 472 aa, Homo sapiens
MALPVTALLLPLALLLHAARPQVQLVQSGAEVKKPGASVKVSCKASGYTFTDYGMDWVRQAPGQGLEWMGYISGGTGRTAYNEKFQGRVTMTRDTSTSTVYMELSSLRSEDTAVYYCARYGANDGFDIWGQGTLVTVSSGGGGSGGGGSGGGGSDIQMTQSPSSLSASVGDRVTITCRASQDISSYLNWYQQKPGKAPKLLIYGASRLYTGVPSRFSGSGSGTDFTLTISSLQPEDFATYYCQQGYNTPYTFGGGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 109 - protein, 472 aa, Homo sapiens
MALPVTALLLPLALLLHAARPQVQLVQSGAEVKKPGASVKVSCKASGYTFTHYAMDWVRQAPGQGLEWMGGISPQSGGTSYNEKFQGRVTMTRDTSTSTVYMELSSLRSEDTAVYYCARVHGDYHFDYWGQGTLVTVSSGGGGSGGGGSGGGGSDIQMTQSPSSLSASVGDRVTITCQASQDVSTDLAWYQQKPGKAPKLLIYHASSLESGVPSRFSGSGSGTDFTLTISSLQPEDFATYYCQQYYNYPLTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 110 - protein, 472 aa, Homo sapiens
MALPVTALLLPLALLLHAARPEVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARPDHGDFMDVWGQGTLVTVSSGGGGSGGGGSGGGGSDIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 111 - protein, 473 aa, Homo sapiens
MALPVTALLLPLALLLHAARPQVQLVQSGAEVKKPGASVKVSCKASGYTFTDYDMSWVRQAPGQGLEWMGVISSSGGSTTYNEKFQGRVTMTRDTSTSTVYMELSSLRSEDTAVYYCARSPMNEDWFDYWGQGTLVTVSSGGGGSGGGGSGGGGSDIQMTQSPSSLSASVGDRVTITCKASQGVSTWLNWYQQKPGKAPKLLIYVASRLESGVPSRFSGSGSGTDFTLTISSLQPEDFATYYCQQSSFLPITFGGGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 112 - protein, 472 aa, Homo sapiens
MALPVTALLLPLALLLHAARPQVQLVQSGAEVKKPGASVKVSCKASGYTFTHYAMSWVRQAPGQGLEWMGGIYGGGDSTNYNEKFQGRVTMTRDTSTSTVYMELSSLRSEDTAVYYCAKASWEDTFDYWGQGTLVTVSSGGGGSGGGGSGGGGSDIQMTQSPSSLSASVGDRVTITCKASQDIRNYLAWYQQKPGKAPKLLIYGASSLASGVPSRFSGSGSGTDFTLTISSLQPEDFATYYCQQSYNYPLTFGQGTKVEIKLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 113 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 114 - protein, 42 aa, Homo sapiens
KRGRKKLLYIFKQPFMRPVQTTQEEDGCSCRFPEEEEGGCEL

SEQ ID NO: 115 - protein, 28 aa, Homo sapiens
IYIWAPLAGTCGVLLLSLVITLYCNHRN

SEQ ID NO: 116 - protein, 45 aa, Homo sapiens
TTTPAPRPPTPAPTIASQPLSLRPEACRPAAGGAVHTRGLDFACD

SEQ ID NO: 117 - protein, 15 aa, Homo sapiens
GGGGSGGGGSGGGGS

SEQ ID NO: 118 - protein, 20 aa, Homo sapiens
GGGGSGGGGSGGGGSGGGGS

SEQ ID NO: 119 - protein, 21 aa, Homo sapiens
MALPVTALLLPLALLLHAARP

SEQ ID NO: 120 - protein, 41 aa, Homo sapiens
RSKRSRLLHSDYMNMTPRRPGPTRKHYQPYAPPRDFAAYRS

SEQ ID NO: 121 - protein, 27 aa, Homo sapiens
FWVLVVVGGVLACYSLLVTVAFIIFWV

SEQ ID NO: 122 - protein, 30 aa, Homo sapiens
LDNEKSNGTIIHVKGKHLCPSPLFPGPSKP

SEQ ID NO: 123 - protein, 11 aa, Homo sapiens
SGYTFTRYTMH

SEQ ID NO: 124 - protein, 17 aa, Homo sapiens
YINPSRGYTNYNQKFKD

SEQ ID NO: 125 - protein, 8 aa, Homo sapiens
YYDDHYCL

SEQ ID NO: 126 - protein, 10 aa, Homo sapiens
SASSSVSYMN

SEQ ID NO: 127 - protein, 7 aa, Homo sapiens
DTSKLAS

SEQ ID NO: 128 - protein, 8 aa, Homo sapiens
QQWSSNPF

SEQ ID NO: 129 - protein, 494 aa, Homo sapiens
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 130 - protein, 477 aa, Homo sapiens
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 131 - protein, 492 aa, Homo sapiens
MALPVTALLLPLALLLHAARPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKRTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 132 - protein, 475 aa, Homo sapiens
MALPVTALLLPLALLLHAARPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKRLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 133 - protein, 245 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 134 - protein, 243 aa, Homo sapiens
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKR

SEQ ID NO: 135 - protein, 122 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 136 - protein, 120 aa, Homo sapiens
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSS

SEQ ID NO: 137 - protein, 5 aa, Homo sapiens
SYYWS

SEQ ID NO: 138 - protein, 5 aa, Homo sapiens
SYFIH

SEQ ID NO: 139 - protein, 16 aa, Homo sapiens
YIYYSGSTNYNPSLKS

SEQ ID NO: 140 - protein, 17 aa, Homo sapiens
FINPYNDGSKYNEKFKG

SEQ ID NO: 141 - protein, 14 aa, Homo sapiens
LVYCGGDCYSGFDY

SEQ ID NO: 142 - protein, 11 aa, Homo sapiens
DDGYYGYAMDY

SEQ ID NO: 143 - protein, 108 aa, Homo sapiens
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 144 - protein, 108 aa, Homo sapiens
DIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKR

SEQ ID NO: 145 - protein, 11 aa, Homo sapiens
QASQDINNFLN

SEQ ID NO: 146 - protein, 11 aa, Homo sapiens
RATQELSGYLS

SEQ ID NO: 147 - protein, 7 aa, Homo sapiens
DASNLET

SEQ ID NO: 148 - protein, 7 aa, Homo sapiens
AASTLDS

SEQ ID NO: 149 - protein, 9 aa, Homo sapiens
QQYGNLPFT

SEQ ID NO: 150 - protein, 9 aa, Homo sapiens
LQYAIYPYT

(19) United States
(12) Patent Application Publication
(10) Pub. No.: US 2026/0337276 A1
(43) Pub. Date: Oct. 29, 2026

(54) CLL1 BINDING MOLECULES

(71) Applicant: CODING BIO LIMITED, Oxford (GB)

(72) Inventors: Simon BORNSCHEIN, Oxford (GB); Youlia LAMPI, Oxford (GB)

(21) Appl. No.: 19/115,629

(22) PCT Filed: Sep. 26, 2023
(86) PCT No.: PCT/GB2023/052497

§ 371 Date: Mar. 26, 2025

(30) Foreign Application Priority Data
Sep. 27, 2022 (GB) 2214132.9

(51) Int. Cl. C07K 16/28; A61K 38/00; A61K 40/11; A61K 40/31; A61K 40/33; A61K 40/41; A61K 47/68; A61K 51/10; C07K 14/705

(52) U.S. Cl. CPC C07K 16/2851; A61K 40/11; A61K 40/31; A61K 40/33; A61K 40/41; A61K 47/6849; A61K 51/1027; C07K 14/7051; C07K 14/70517; C07K 14/70521; C07K 16/2809; A61K 38/00; C07K 2317/622; C07K 2319/02; C07K 2319/03

(57) ABSTRACT

The present disclosure relates to a novel class of C-type lectin-like molecule 1 (CLL1) binding molecules, in particular to anti-CLL1 antibodies and antigen binding fragments. This disclosure also relates to the synthetic molecules comprising the anti-CLL1 antibodies and antigen binding fragments, such as chimeric antigen receptors (CARs), bispecific T-Cell engagers (BiTEs) and antibody-drug conjugates (ADCs), and use of the anti-CLL1 antibodies and antigen binding fragments, or synthetic molecules comprising them, in the treatment of disease.

### FIELD OF THE INVENTION

The present invention relates to C-type lectin-like molecule 1 (CLL1) binding molecules, in particular to anti-CLL1 antibodies and antigen binding fragments. The present invention further relates to recombinant molecules comprising said CLL1 binding molecules, such as, chimeric antigen receptors (CARs), BiTEs and ADCs. Another object of the present invention is an engineered immune cell, suitably, a regulatory T cell comprising said CAR. Uses thereof in therapeutic methods of said antibody, said recombinant molecule and said engineered immune cell are further described.

### BACKGROUND OF THE INVENTION

The C-type lectin-like molecule 1 (CLL1) glycoprotein (also known as CLEC12A, DCAL2, MICL, KLRL1) is a type II transmembrane protein comprising a single C-type lectin-like domain, a transmembrane domain, a stalk region, and a cytoplasmic tail containing an immunoreceptor tyrosine-based inhibitory motif (ITIM) (https://www.uniprot.org/uniprotkb/Q5QGZ9/entry; Marshall et al. 2006. Human CLL1 is differentially glycosylated and is down-regulated following cellular activation. Eur. J. Immunol., 36, 2159-2169).

CLL1 functions as a cell surface receptor and is expressed on myeloid lineage cells. The cytoplasmic ITIM motif is a conserved sequence of amino acids, commonly found in a number of immune-function related receptors, which contains a tyrosine residue that may be phosphorylated and thereby effect a signalling cascade which is thought to be important for regulating cellular maturation and activation. Importantly, while CLL1 is expressed on myeloid lineage cells (pre-activation), it shows limited expression in other cell lineages and tissues, and it is not expressed on haematopoietic stem cells (HSCs). Furthermore, CLL1 has been found to be expressed in more than 80% of acute myeloid leukaemia (AML) patients. Therefore, CLL1 represents a good target for immunotherapy as it appears to be an attractive protein to discriminate between AML and non-AML cells.

AML typically affects middle aged and older adults and represents approximately 1% of new cancer cases every year in the USA (Cancer Stat Facts: Leukemia-Acute Myeloid Leukemia (AML). Available online: https://seer.cancer.gov/statfacts/html/amyl.html (accessed on 5 Sep. 2022)). Historically, AML has been challenging to treat and is associated with a high mortality rate. For example, in the USA, AML has a 5-year relative survival rate of 30.5% (Cancer Stat Facts: Leukemia-Acute Myeloid Leukemia (AML). Available online: https://seer.cancer.gov/statfacts/html/amyl.html (accessed on 5 Sep. 2022)). One reason for this difficulty is that AML has many antigens in common with haematopoietic stem cells (HSCs). Hence, in most instances of AML new therapeutic methods, such as immunotherapy, have been unable to specifically target cancerous cells.

CLL1 is also implicated with other cancers and diseases including but not limited to: AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN), or combinations thereof.

CLL1 may additionally play a role in inflammatory or autoimmune diseases such as rheumatoid arthritis, psoriasis, allergies, asthma, Crohn's disease, IBD, IBS, fibromyalga, mastocytosis, and Celiac disease.

Therapeutic treatments that make use of the immune system or immune cells are known as immunotherapy treatments. In some cases, immunotherapy treatment relies on antibodies to an antigen on a target cell triggering or promoting cell death or inhibiting cell-division. Antigens may be targeted using antibody or antigen binding fragments, such as a single chain variable fragment (scFv). Examples of antibodies that target CLL1 have been previously described e.g. U.S. Pat. No. 9,163,090 B2.

The antibody or antigen binding domain can be linked or conjugated to a synthetic molecule, such as a synthetic molecule that binds to a T-cell antigen, suitably via a flexible linker, such as a flexible peptide linker to form a bi-specific T-cell engager (BiTE), or a dual-affinity retargeting antibody (DART) or other bi-specific, tri-specific or multi-specific T-cell engagers.

Alternatively, such antibodies or antigen binding fragments may be linked or conjugated with a synthetic molecule such as a label, cytotoxic agent (antibody-drug conjugates or ADCs), or therapeutic isotope.

A specific type of immunotherapy is chimeric antigen receptor (CAR) T-cell therapy. CAR T-cell therapy consists of obtaining T-cells from a patient and incorporating an engineered receptor to recognise an antigen found on the cancerous cells. The resulting CAR T-cells are then incorporated back into the patient, where they may then act to mount an immune response against the antigen i.e. against the cancerous cells, and kill the cancerous cells.

In this case, an antibody or antigen binding fragment can be linked or conjugated with a synthetic molecule comprising other protein domains, such as hinge domains, transmembrane domains, co-stimulation domains and activation domains to form a CAR that is capable of effecting an immune response towards the antigen-containing cell. This CAR is then expressed in a T-cell.

Examples of CARs that target CLL1 have been previously described e.g. U.S. Pat. No. 10,568,947 B2 and U.S. Pat. No. 10,597,456 B2 and Zhang et al. Clin Cancer Res. 2021; 27 (13): 3549-3555,

Two recent reports have claimed success of CLL1-based CAR T cells in patients with secondary AML (Zhang et al. Front Oncol. 2020; 10:685; and Fang et al. Blood. 2018; 132:901), thereby highlighting potential for use in AML and other cancers.

The nature of the binding between the CAR and the target antigen, and the associated activation of the T-cell as a result of this binding via other functional domains, is critical to the effectiveness of the CAR T-cell therapy.

Producing CARs that demonstrate high activation from low levels of CLL1 is important due to the known variable level of CLL1 expression in cells, and the potential for antigen escape during immunotherapy treatment.

Previous anti-CLL1 CARs have shown varying levels of effectiveness at eliciting an immune response and killing cancer cells. The reasons for this remain unknown, but it may be related to a low binding affinity of the antibody or antibody derivative portion of the CAR, which results in poor binding to CLL1 and weaker activation of the immune response. Alternatively, this may be due to the lack of optimisation or co-optimisation of the remaining functional domains of the CAR, which also affect T-cell activation.

Therefore, there exists a need for novel and improved antibodies, or antigen binding fragments suitable for treating CLL1-associated diseases and disorders.

### SUMMARY OF THE INVENTION

In a first aspect of the invention, there is provided an antibody, or antigen binding fragment, which binds to CLL1 selected from the group consisting of:
Antibody 5, comprising:
   (a) a heavy chain variable region comprising,
      (i) a CDR1 of SEQ ID NO:27,
      (ii) a CDR2 of SEQ ID NO:37, and
      (iii) a CDR3 of SEQ ID NO:47; and
   (b) a light chain variable region comprising,
      (i) a CDR1 of SEQ ID NO:67,
      (ii) a CDR2 of SEQ ID NO:77, and
      (iii) a CDR3 of SEQ ID NO:87;
and,
Antibody 1, comprising:
   (a) a heavy chain variable region comprising,
      (i) a CDR1 of SEQ ID NO:23,
      (ii) a CDR2 of SEQ ID NO:33, and
      (iii) a CDR3 of SEQ ID NO:43; and
   (b) a light chain variable region comprising,
      (i) a CDR1 of SEQ ID NO:63,
      (ii) a CDR2 of SEQ ID NO:73, and
      (iii) a CDR3 of SEQ ID NO:83;
and,
Antibody 2, comprising:
   (a) a heavy chain variable region comprising,
      (i) a CDR1 of SEQ ID NO:24,
      (ii) a CDR2 of SEQ ID NO:34, and
      (iii) a CDR3 of SEQ ID NO:44; and
   (b) a light chain variable region comprising,
      (i) a CDR1 of SEQ ID NO:64,
      (ii) a CDR2 of SEQ ID NO:74, and
      (iii) a CDR3 of SEQ ID NO:84;
and,
Antibody 3, comprising;
   (a) a heavy chain variable region comprising,
      (i) a CDR1 of SEQ ID NO:25,
      (ii) a CDR2 of SEQ ID NO:35, and
      (iii) a CDR3 of SEQ ID NO:45; and
   (b) a light chain variable region comprising,
      (i) a CDR1 of SEQ ID NO:65,
      (ii) a CDR2 of SEQ ID NO:75, and
      (iii) a CDR3 of SEQ ID NO:85;
and,
Antibody 4, comprising:
   (a) a heavy chain variable region comprising,
      (i) a CDR1 of SEQ ID NO:26,
      (ii) a CDR2 of SEQ ID NO:36, and
      (iii) a CDR3 of SEQ ID NO:46; and
   (b) a light chain variable region comprising,
      (i) a CDR1 of SEQ ID NO:66,
      (ii) a CDR2 of SEQ ID NO:76, and
      (iii) a CDR3 of SEQ ID NO:86;
and,
Antibody 6, comprising:
   (a) a heavy chain variable region comprising,
      (i) a CDR1 of SEQ ID NO:28,
      (ii) a CDR2 of SEQ ID NO:38, and
      (iii) a CDR3 of SEQ ID NO:48; and
   (b) a light chain variable region comprising,
      (i) a CDR1 of SEQ ID NO:68,
      (ii) a CDR2 of SEQ ID NO:78, and
      (iii) a CDR3 of SEQ ID NO:88;
and,
Antibody 7, comprising:
   (a) a heavy chain variable region comprising,
      (i) a CDR1 of SEQ ID NO:29,
      (ii) a CDR2 of SEQ ID NO:39, and
      (iii) a CDR3 of SEQ ID NO:49; and
   (b) a light chain variable region comprising,
      (i) a CDR1 of SEQ ID NO:69,
      (ii) a CDR2 of SEQ ID NO:79, and
      (iii) a CDR3 of SEQ ID NO:89;
and,
Antibody 8, comprising:
   (a) a heavy chain variable region comprising,
      (i) a CDR1 of SEQ ID NO:30,
      (ii) a CDR2 of SEQ ID NO:40, and
      (iii) a CDR3 of SEQ ID NO:50; and
   (b) a light chain variable region comprising,
      (i) a CDR1 of SEQ ID NO:70,
      (ii) a CDR2 of SEQ ID NO:80, and
      (iii) a CDR3 of SEQ ID NO:90;
and,
Antibody 9, comprising:
   (a) a heavy chain variable region comprising,
      (i) a CDR1 of SEQ ID NO:31,
      (ii) a CDR2 of SEQ ID NO:41, and
      (iii) a CDR3 of SEQ ID NO:51; and
   (b) a light chain variable region comprising,
      (i) a CDR1 of SEQ ID NO:71,
      (ii) a CDR2 of SEQ ID NO:81, and
      (iii) a CDR3 of SEQ ID NO:91;
and,
Antibody 10, comprising:
   (a) a heavy chain variable region comprising,
      (i) a CDR1 of SEQ ID NO:32,
      (ii) a CDR2 of SEQ ID NO:42, and
      (iii) a CDR3 of SEQ ID NO:52; and
   (b) a light chain variable region comprising,
      (i) a CDR1 of SEQ ID NO:72,
      (ii) a CDR2 of SEQ ID NO:82, and
      (iii) a CDR3 of SEQ ID NO:92.

In embodiments, the antibody, or antigen binding fragment, which binds to CLL1 selected from the group consisting of: Antibody 4, Antibody 5 and Antibody 7. Suitably, the antibody, or antigen binding fragment, which binds to CLL1 is Antibody 5.

In embodiments, the antibody or antigen binding fragment is comprised in a single chain variable fragment (scFv). Suitably, the scFv is selected from the group consisting of: SEQ ID NOs 3 to 12. Suitably, the scFv is selected from the group consisting of: SEQ ID Nos 6, 7 and 9. Suitably, the scFv is SEQ ID No. 7.

In embodiments, the antibody or antigen binding fragment is humanised.

In further embodiments, the antibody or antigen binding fragment is conjugated to a synthetic molecule.

Suitably, the antibody or antigen binding fragment is a chimeric antigen receptor and the synthetic molecule comprises a transmembrane region and an intracellular T-cell receptor signaling domain. Suitably, the transmembrane region is from CD8A or CD28 and/or the intracellular signaling domain is from CD247 (CD3-zeta). Suitably, the chimeric antigen receptor further comprises an intracellular domain of a costimulatory protein receptor. Suitably, the costimulatory domain is from CD137 or CD28.

Suitably, the antibody or antigen binding fragment is a bi-specific T-cell engager (BiTE) and the synthetic molecule comprises an antigen binding domain which binds to a T-cell antigen. Suitably, the antigen binding domain comprises an antibody fragment that specifically binds CD3. Suitably, the antibody fragment that specifically binds CD3 comprises:
(a) a heavy chain variable region comprising,
   (i) a CDR1 of SEQ ID NO:123,
   (ii) a CDR2 of SEQ ID NO:124, and
   (iii) a CDR3 of SEQ ID NO:125; and
(b) a light chain variable region comprising,
   (i) a CDR1 of SEQ ID NO:126,
   (ii) a CDR2 of SEQ ID NO:127, and
   (iii) a CDR3 of SEQ ID NO:128.

In embodiments, the antibody is an antigen-drug conjugate and the synthetic molecules is a cytotoxic drug or a therapeutic radioisotope.

In a second aspect, the invention provides a vector encoding the antibody, or antigen binding fragment, of the first aspect of the invention.

In a third aspect, the invention provides a pharmaceutical composition comprising the antibody or antigen binding fragment, of the first aspect of the invention and a pharmaceutically acceptable carrier.

In a fourth aspect, the invention provides a chimeric antigen receptor comprising:
a) The antibody or antigen binding fragment as claimed in any one of the first aspect;
b) A transmembrane region;
c) An intracellular T-cell receptor signaling domain.

In embodiments, the transmembrane region and intracellular T-cell receptor signaling domain of the chimeric antigen receptor of the fourth aspect of the invention are from CD3 zeta. In embodiments, the chimeric antigen receptor further comprises a transmembrane domain or an intracellular signaling domain of a costimulatory protein receptor. In embodiments, the chimeric antigen receptor further comprises a hinge region to create flexibility or increased intercellular spacing.

In embodiments the CAR of the fourth aspect of the invention comprises:
a. a human CD247 (CD3-zeta) cytoplasmic domain (SEQ ID NO:113), a human CD137 co-stimulatory domain (SEQ ID NO:114); a human CD8A transmembrane domain (SEQ ID NO:115), a human CD8A hinge domain (SEQ ID NO:116); antibody or antigen binding fragment of the first aspect; and an CD8A extracellular signal peptide (SEQ ID NO: 119); or
b. a human CD247 (CD3-zeta) cytoplasmic domain (SEQ ID NO:113), a human CD28 co-stimulatory domain (SEQ ID NO:120); a human CD28 transmembrane domain (SEQ ID NO: 121), a human CD28 hinge domain (SEQ ID NO:122); antibody or antigen binding fragment of the first aspect; and an CD8A extracellular signal peptide (SEQ ID NO: 119).

In embodiments, the CAR has a sequence selected from SEQ ID NOS: 93 to 102. Suitably, the CAR has a sequence selected from SEQ ID Nos. 96; 97 and 99. Suitably, the CAR has a sequence of SEQ ID No. 97.

In a fifth aspect, the invention provides a recombinant T cell comprising the chimeric antigen receptor of the fourth aspect of the invention.

In a sixth aspect of the invention there is provided a bispecific T-cell engager comprising:
a) The antibody or antigen binding fragment of the first aspect;
b) An antigen binding domain which binds to a T-cell antigen;
c) A linker joining (a) and (b).

In embodiments, the antigen binding domain comprises an antigen binding fragment that specifically binds CD3.

In embodiments, the antibody fragment that specifically binds CD3 comprises:
(a) a heavy chain variable region comprising,
   (i) a CDR1 of SEQ ID NO:123,
   (ii) a CDR2 of SEQ ID NO:124, and
   (iii) a CDR3 of SEQ ID NO:125; and
(b) a light chain variable region comprising,
   (i) a CDR1 of SEQ ID NO:126,
   (ii) a CDR2 of SEQ ID NO:127, and
   (iii) a CDR3 of SEQ ID NO:128;
In embodiments, the linker is a flexible linker. Suitably, the linker is a peptide or protein linker. Suitably, the linker is a flexible peptide linker covalently attached to (a) and (b).

In a seventh aspect of the invention there is provided an antibody-drug conjugate comprising:
a) The antibody or antigen binding fragment of the first aspect;
b) A synthetic molecule;
c) A linker joining (a) and (b).

In embodiments, the synthetic molecule is a cytotoxic drug. In embodiments, the linker is cleavable or non-cleavable. Suitably, the linker is a chemical linker that separately covalently attaches to (a) and (b). In an eighth aspect of the invention there is provided an antibody, or antigen binding fragment, of any the first aspect, a pharmaceutical composition of the third aspect, a chimeric antigen receptor of the fourth aspect, a recombinant T-cell of the fifth aspect, a bi-specific T-cell engager of sixth aspect or a antibody-drug conjugate of the seventh aspect for use as a medicament.

In a ninth aspect of the invention there is provided an antibody, or antigen binding fragment, of any the first aspect, a pharmaceutical composition of the third aspect, a chimeric antigen receptor of the fourth aspect, a recombinant T-cell of the fifth aspect, a bi-specific T-cell engager of sixth aspect or a antibody-drug conjugate of the seventh aspect for use in killing or inhibiting the growth of cells expressing CLL1.

In a tenth aspect of the invention there is provided an antibody, or antigen binding fragment, of any the first aspect, a pharmaceutical composition of the third aspect, a chimeric antigen receptor of the fourth aspect, a recombinant T-cell of the fifth aspect, a bi-specific T-cell engager of sixth aspect or a antibody-drug conjugate of the seventh aspect for use in the treatment of a disease or disorder related to an elevated expression of CLL1.

In an eleventh aspect of the invention there is provided an antibody, or antigen binding fragment, of any the first aspect, a pharmaceutical composition of the third aspect, a chimeric antigen receptor of the fourth aspect, a recombinant T-cell of the fifth aspect, a bi-specific T-cell engager of sixth aspect or a antibody-drug conjugate of the seventh aspect for use in medicine.

In an twelfth aspect of the invention there is provided an antibody, or antigen binding fragment, of any the first aspect, a pharmaceutical composition of the third aspect, a chimeric antigen receptor of the fourth aspect, a recombinant T-cell of the fifth aspect, a bi-specific T-cell engager of sixth aspect or a antibody-drug conjugate of the seventh aspect for use in the treatment of a disease or disorder, wherein the disease or disorder is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease.

In a thirteenth aspect of the invention there is provided a method of killing or inhibiting the growth of cells expressing CLL1 in a subject, said method comprising administering an effective amount of the antibody, or antigen binding fragment, of the first aspect, the pharmaceutical composition of the third aspect, the chimeric antigen receptor of the fourth aspect, the recombinant T-cell of the fifth aspect, the bi-specific T-cell engager of sixth aspect or the antibody-drug conjugate of the seventh aspect. In a fourteenth aspect of the invention there is provided a method of treating a disease or condition associated with the expression of CLL1, said method comprising administering to a patient in need thereof an effective amount of an the antibody, or antigen binding fragment, of the first aspect, the pharmaceutical composition of the third aspect, the chimeric antigen receptor of the fourth aspect, the recombinant T-cell of the fifth aspect, the bi-specific T-cell engager of sixth aspect or the antibody-drug conjugate of the seventh aspect.

In embodiments of the fourteenth aspect of the invention, the disease or condition is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease.

### BRIEF DESCRIPTION OF THE FIGURES

FIG. 1 shows (A) an embodiment of the general structure of the antibody chain or antigen binding fragment (scFv) of the present invention, with (i) a first VH chain, a linker and a VL chain as defined in the exemplified examples, and an alternative antibody configuration that is also encompassed by the present invention, with (ii) a first VL chain, a linker and a VH chain; (B) an example chimeric antigen receptor scaffold 1 of an embodiment of the present invention comprising a CD8A hinge and CD8A TM region (‘CD8A-CAR’); and (C) exemplified example chimeric antigen receptor scaffold 2 of an embodiment of the present invention comprising a CD28 hinge and CD28 TM region (‘CD28-CAR’). The ‘CD8A-CAR’ and the ‘CD28-CAR’ are embodiments of another aspect of the invention.

FIG. 2 shows median fluorescence intensity (MFI) from primary human T cells transduced with the different CLL1-CAR constructs. The median fluorescence intensity indicates the affinity/avidity with which the T cells with different CARs bind to CLL1. Following transduction, the cells were expanded for eight days and stained with CLL1-Fc fusion protein. In a second step, cells were stained with PE-conjugated anti-Fc and an APC-conjugated anti-CD34 antibodies. Median fluorescence intensity of CLL1-Fc staining is shown for CD34+ CAR-T cells. FIG. 2A shows a first T cell donor and FIG. 2B shows a second experiment, with a different T cell donor.

FIG. 3 shows the expansion of human primary T cells transduced with different CLL1 CAR constructs and their average fold expansion between day 4 and day 8 of the CAR-T manufacturing process. Cells were counted using a LUNA FL cell counter (Logos Biosystems) and fluorescence live/dead dye.

FIG. 4 shows % CD69 upregulation by human primary T cells transduced with the different CLL1-CAR constructs or mock transduced T cells, expanded for eight days and co-cultured for 24 h with CLL1-expressing cancer cell line (HL60 cell line). Flow cytometry was performed to separate CLL1-CAR-T cells from the cancer cells. CD69 up-regulation on CD3+ and CD34+ CAR-T was assessed by flow cytometry

FIG. 5 shows killing capacity of CLL1-CAR-T cells against CLL1-expressing HL60 cancer cell line. CAR-T cells, transduced with anti-CLL1 CAR constructs or mock transduced T cells, were co-cultured with HL60 cancer cells at a 1:1 ratio for 24 h. As a control, HL60 cells were cultured in the absence of T cells. After 24 h HL60 cells were quantified by flow cytometry and percentage of killed HL60 cells was calculated, relative to HL60 cells cultured alone.

FIG. 6 shows potency of CLL1-CAR-T cells after stimulation with CLL1-expressing HL60 cancer cell line or non-stimulated controls. For each label, the bar on the left indicates CLL1-CAR-T cells that are stimulated with HL60, and the bar on the right indicates non-stimulated controls. CAR-T cells, transduced with anti-CLL1 CAR constructs or mock transduced T cells, were co-cultured with HL60 cancer cells at a 1:1 ratio for 24 h. After 24 h supernatant was assessed for IFNg production via cytokine ELISA.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting.

All references cited herein are incorporated by reference in their entirety. Unless otherwise defined, all technical and scientific terms used herein have the same meaning as commonly understood by one of ordinary skill in the art to which this invention belongs.

Prior to further setting forth the invention, a number of definitions are provided that will assist in the understanding of the invention.

The articles ‘a’, ‘an’ and ‘the’ are used to refer to one or to more than one (i.e. to at least one) of the grammatical object of the article.

As used herein, the term ‘comprising’ means any of the recited elements are necessarily included and other elements may optionally be included as well. ‘Consisting essentially of’ means any recited elements are necessarily included, elements which would materially affect the basic and novel characteristics of the listed elements are excluded, and other elements may optionally be included. ‘Consisting of’ means that all elements other than those listed are excluded. Embodiments defined by each of these terms are within the scope of this invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

The term ‘antibody’ refers to all isotypes of immunoglobulins (IgG, IgA, IgE, IgM, IgD, and IgY) including various monomeric, polymeric and chimeric forms, unless otherwise specified. Specifically encompassed by the term ‘antibody’ are polyclonal antibodies, monoclonal antibodies (mAbs), single domain antibodies, human (FHVH) or heavy-chain antibodies found in camelids (VHH) and antibody-like polypeptides, such as chimeric antibodies and humanized antibodies. The term ‘monoclonal antibody’ refers to an antibody produced by a single clone of cells or cell line and consisting of identical antibodies.

As used herein, the term ‘antigen-binding fragments’ refers to any protein structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the V_{L}, V_{H}, C_{L} and C_{H}1 domains, or a monovalent antibody as described in WO2007059782, bivalent fragments comprising two Fab fragments linked by a disulfide bridge at the hinge region, a Fd fragment consisting essentially of the V_{H} and C_{H}1 domains; a Fv fragment consisting essentially of the V_{L} and V_{H} domains of a single arm of an antibody, a dAb fragment (Ward et al., Nature 341, 544-546 (1989)), which consists essentially of a V_{H} domain and also called domain antibodies (Holt et al; Trends Biotechnol. 2003 November; 21 (11): 484-90); camelid or nanobodies (Revets et al; Expert Opin Biol Ther. 2005 January; 5 (1): 111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase ‘an antibody or antigen-binding fragment’ may be used to denote that a given antigen binding fragment incorporates one or more amino acid segments of the antibody referred to in the phrase.

As used herein the term ‘antigen binding domain’ refers to a peptide sequence that is intended or able to bind a target of interest. In examples, the antigen binding domain is an antigen-binding fragment as defined above. All types of antigen binding domains are encompassed by the present invention. Examples of some antigen binding domains are scFvs, VHH single domain antibodies or nanobodies, and antigen binding fragments.

An ‘scFv’ or ‘single chain variable fragment’ as used herein, refers to a type of antigen binding domain. Typically, an scFv is a fusion of the variable regions of the heavy (V_{H}) and light chains (V_{L}) of an antibody for a given target connected by a short linker. The VH and VL regions may be in any order around the linker, for example, the scFv may have (i) a first VH chain, a linker and a VL chain or (ii) a first V_{L} chain, a linker and a VH chain. As it is generally accepted that both versions would lead to similar activity, both are encompassed by the present disclosure even in the event that only one is exemplified. Antigen binding domains may comprise ‘CDRs’ or ‘complementarity determining regions’ which are predominantly responsible for target binding. On a typical antibody, multiple CDRs exist and may be selected or varied independently to achieve multiple points of diversity. A ‘recognition sequence’ refers to the nucleic acid sequence encoding one or more antigen binding domains.

‘Specific binding’ or ‘immunospecific binding’ or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules.

Typically, an antibody binds to a cognate antigen with a Kd of less than about 1×10⁻⁸ M, as measured by a surface plasmon resonance assay or a cell binding assay. Phrases such as ‘[antigen]-specific’ antibody (e.g. CLL1-specific antibody) are meant to convey that the recited antibody specifically binds the recited antigen.

As used herein, the term ‘isolated’ means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been isolated thus include nucleic acids and proteins purified by standard purification methods. Isolated nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An ‘isolated’ antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to CLL1 is substantially free of antibodies that specifically bind antigens other than CLL1). An isolated antibody that specifically binds to an epitope, isoform or variant of CLL1 may, however, have cross-reactivity to other related antigens, for instance from other species (such as CLL1 species homologs).

As used herein, the term ‘chimeric antigen receptor’ or ‘CAR’ refers to transmembrane receptor that has been engineered to target or bind to a non-native substrate or antigen. In this way the intracellular signalling of the receptor may triggered by binding of a non-native substrate or antigen. Typically, the term CAR refers to a chimeric receptor (i.e. a receptor composed of two or more parts from different sources) that has at least a binding moiety or recognition sequence with a specificity for a target such as an antigen or protein and an intracellular signaling domain that can invoke a signal in the cell in which the CAR is expressed (e.g. a CD247 (CD3 zeta) chain). In embodiments, a ‘chimeric antigen receptor’ or ‘CAR’ is formed of at least three domains: an extracellular antigen binding domain (as defined elsewhere herein), a transmembrane domain and an intracellular domain. A hinge domain between the antigen binding domain and the transmembrane domain is often used to improve recognition of the target. A costimulatory domain may optionally be present in the intracellular domain to modulate the response. To be functional, the domains of the CAR must be ordered correctly. CARs are often used on T-cells (to produce ‘CAR T-cells’) to effect recognition and elicit an appropriate intracellular response which both binds the T-cell to a target cell and triggers the innate immune response of the T-cell, typically causing lysis of the target cell. Such cells have found use in therapy.

A ‘transmembrane domain’ or ‘TM domain’ as used herein is any membrane-spanning protein domain. Suitably, the TM domain in a CAR is derived from a known transmembrane protein sequence. However, it can also be artificially designed. A ‘transmembrane sequence’ refers to the nucleic acid sequence encoding a transmembrane domain.

As used herein the term ‘hinge domain’ refers to a peptide sequence that connects the antigen binding domain and transmembrane region of a CAR. The hinge domain is located between the antigen binding fragment and the T cell plasma membrane (Moritz D, et al. Gene Ther. 1995; 2(8):539-46)

The term ‘signaling domain’ or ‘intracellular domain’ or ‘intracellular signaling domain’ as used herein refers to a moiety that can transmit a signal in a cell, for example an immune cell. The signaling domain typically comprises a domain derived from a receptor that signals by itself in immune cells, such as the T Cell Receptor (TCR) complex or the Fc receptor or DAP10/DAP12 receptors. Additionally, it may contain a costimulatory domain (i.e. a domain derived from a receptor that is required in addition to the TCR to obtain full activation of T cells). The costimulatory domain can be from an activating costimulatory receptor or from an inhibitory costimulatory receptor. An ‘intracellular domain sequence’ refers to the nucleic acid sequence encoding an intracellular signaling domain.

As used herein, the terms ‘bi-specific’, ‘tri-specific’ or ‘multi-specific’ refer to an antibody molecule (i.e. an antibody or antigen binding fragment conjugated to a synthetic molecule) that comprises one or more further antigen binding domains such that the antibody molecule can have specificity for more than one antigen.

As is known in the art, as used herein, the term ‘bispecific T-cell engager’ or ‘BiTE’ is used synonymously to refer to a single polypeptide chain molecule having two antigen binding domains, one of which binds to a T-cell antigen (e.g., CD3) and the second of which binds to an antigen present on the surface of a target cell (e.g. CLL1), these two antigen binding domains separated by a linker, specifically a flexible linker, more specifically a flexible protein linker (WO 05/061547; Baeuerle, et al. (2008) Drugs of the Future 33:137-147; Bargou, et al. (2008) Science 321:974-977). BiTE antibodies have been constructed to various target antigens including CD19, EpCAM, Her2/neu, EGFR, CD66e (or CEA, CEACAM5), CD33, EphA2, and MCSP (or HMW-MAA) (Baeuerle, et al. (2009) Curr. Opin. Mol. Ther. 11:22-30). Key hallmarks of BiTE antibodies that, in their combination, distinguish them from other bispecific antibody constructs, include a high potency of redirected lysis with EC50 values ranging from 0.1 to 50 μmol/L (2-1,000 μg/mL) (Baeuerle, et al. (2009) supra); strict target cell-dependent activation of T cells (Brischwein, et al. (2007) J. Immunother. 30:798-807); and support of serial lysis by activated T cells, i.e., activity at low E: T ratios. BiTE antibodies are typically produced as recombinant, glycosylated proteins secreted by higher eukaryotic cell lines. The terms ‘bi-specific T-Cell engager’ and/or ‘BiTE’ as herein defined encompass, but are not limited to, the specific bi-specific T-Cell engager structure encompassed by the trademark ‘BITER’ in the name of Amgen Research (Munich) GmbH.

The term ‘DART™ ’ (Dual Affinity Re-Targeting reagent) diabody refers to an immunoglobulin molecule that comprises at least two polypeptide chains that associate (for example, through a covalent interaction) to form at least two antigen binding domains, which may recognize the same or different antigens. Each of the polypeptide chains of a DART™ diabody comprise an immunoglobulin light chain variable region and an immunoglobulin heavy chain variable region, but these regions do not interact to form an antigen binding domain. Rather, the immunoglobulin heavy chain variable region of one (e.g., the first) of the DART™ diabody polypeptide chains interacts with the immunoglobulin light chain variable region of a different (e.g. the second) DART™ polypeptide chain to form an antigen binding domain. Similarly, the immunoglobulin light chain variable region of one (e.g., the first) of the DART™ diabody polypeptide chains interacts with the immunoglobulin heavy chain variable region of a different (e.g., the second) DART™ diabody polypeptide chain to form an antigen binding domain. DART™ diabodies may be monospecific, bispecific, trispecific, etc., thus being able to simultaneously bind one, two, three or more different antigens (which may be of the same or of different antigens). DART™ diabodies may additionally be monovalent, bivalent, trivalent, tetravalent, pentavalent, hexavelent, etc., thus being able to simultaneously bind one, two, three, four, five, six or more molecules. These two attributes of DART™ diabodies {i.e., degree of specificity and valency may be combined, for example to produce bispecific antibodies {i.e., capable of binding two antigens) that are tetravalent {i.e., capable of binding four sets of epitopes), etc. DART™ diabody molecules are disclosed in PCT applications published as WO 2006/113665, WO 2008/157379, and WO 2010/080538.

As used herein, the term ‘antibody-drug conjugate’ or ‘ADC’ means a biopharmaceutical drug that comprises an antibody or antigen binding fragment, and a drug substance, typically a cytotoxic drug substance, connected via a linker, suitably a covalent, chemical linker.

As used herein, the term ‘high-throughput screening’ refers to any assay or screening methodology that allows for a higher rate of screening than would be achieved by traditional or previous state of the art techniques. Typically, high-throughput screening enables automation to prepare, screen and/or evaluate libraries of test samples in parallel, reproducibly and rapidly. High-throughput screening can also make use of combinatorial or pooled or mixed sample screening strategies, with associated deconvolution of hits. In the context of CAR high throughput screening, the number of samples for test may be of any size larger than that typically used in prior art non-high-throughput methods. For example, the number of samples may be more than 10. Suitably the number of samples may be more than 11, 12, 13, 14, 15, 16, 17, 18, 19, 20, 30, 40, 50, 100, 200 or more.

As used herein, the term ‘recognition sequence library’ refers to a set of one or more antigen binding domain sequences or recognition sequences that may be used for cloning into a CAR construct (wherein a CAR construct comprises all components required for a functioning CAR including one or more antigen binding domains, a hinge domain, a transmembrane domain and an intracellular domain) to prepare a CAR library. An ‘scFv library’ is a recognition sequence library formed of scFv recognition sequences from antibodies. In embodiments, the number of recognition sequences present in the recognition sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term ‘CAR scaffold’ refers to the part of the CAR sequence that comprises the components, parts, modules, domains of the CAR excluding the antigen binding domain, that form the sequence of a CAR in a CAR library. The CAR scaffold is formed of the sequences of the individual components, or groups thereof, and combined with the recognition sequence to form a sequence encoding a CAR in any suitable manner, for example, sequentially, convergently, with the recognition sequence being incorporated at any suitable point, i.e. the recognition sequence may be joined to a single unitary sequence of the CAR scaffold, or may be joined to a component part of the CAR scaffold initially and the full CAR sequence of the recognition sequence and scaffold completed subsequently. The component parts of the CAR scaffold may derive from single sequences leading to one or a small number of CAR scaffolds for addition to a recognition sequence, or the component parts of the CAR scaffold may derive from libraries of one or more of the individual components leading to a CAR scaffold library comprising a plurality of sequences. A CAR scaffold library may be formed by combination, suitably in a combinatorial, or directed, manner of the individual components or component libraries.

As used herein, the term ‘CAR library’ refers to a set of sequences encoding a functional CAR structure, including a recognition domain, such as an scFv, a hinge domain, a transmembrane domain and intracellular domain(s). In embodiments, the number of CARs present in the CAR library may be more than 10. Suitably the number of CARs present in the CAR library may be more than 11, 12, 13, 14, 15, 16, 17, 18, 19, 20, 30, 40, 50, 100, 200 or more. The term ‘CAR library’ may also be used interchangeably to refer to the plasmid or vector, or otherwise modified sequence of the sequence(s) encoding for a functional CAR structure.

As used herein, the term ‘CAR-cell library’ refers to a collection or set of cells expressing CARs on their surface. Each cell in the CAR-cell library may express a single CAR (i.e. express only one CAR encoded by a single sequence), or a single cell in the CAR-cell library may express two or more CARs, each encoded by a different sequence. Suitably, each cell in the CAR-cell library expresses a single CAR or all CARs present in the library. In embodiments, the number of CARs present in the CAR-cell library may be more than 10. Suitably the number of CARs present in the CAR-cell library may be more than 11, 12, 13, 14, 15, 16, 17, 18, 19, 20, 30, 40, 50, 100, 200 or more.

As used herein, the term ‘next generation sequencing’ of ‘NGS’ refers to a catch-all term used to describe a number of different modern sequencing technologies. These technologies allow for sequencing of DNA and RNA much more quickly and cheaply than the previously used Sanger sequencing. Examples are Solex™ sequencing by Illumina™, Roche 454™ sequencing, Ion Torrent™ sequencing and nanopore based sequencing methods (e.g. Oxford Nanopore Technologies™ Grid ION™).

The phrase ‘nucleic acid molecule’ synonymously referred to as ‘nucleotides’ or ‘nucleic acids’ or ‘polynucleotide’ refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, ‘polynucleotide’ refers to triple-stranded regions comprising RNA or DNA or both RNA and DNA. The term polynucleotide also includes DNAs or RNAs containing one or more modified bases and DNAs or RNAs with backbones modified for stability or for other reasons. ‘Modified’ bases include, for example, tritylated bases and unusual bases such as inosine. A variety of modifications may be made to DNA and RNA; thus, ‘polynucleotide’ embraces chemically, enzymatically or metabolically modified forms of polynucleotides as typically found in nature, as well as the chemical forms of DNA and RNA characteristic of viruses and cells. ‘Polynucleotide’ also embraces relatively short nucleic acid chains, often referred to as oligonucleotides.

There are various means by which a nucleic acid sequence may be inserted into a genome, including but not limited to plasmid or vector transfection, transposition and genome editing. All are contemplated for use in the present invention.

As used herein a ‘vector’ is a replicon, such as plasmid, phage, cosmid, or virus in which another nucleic acid segment may be operably inserted so as to bring about the replication or expression of the segment. A ‘transposon’ or ‘transposable elements’ are DNA sequences that can change their position within a genome. ‘Genome editing’ refers to the ability to edit the genome to insert the required sequence, for example using CRISPR-Cas9 genome editing technology.

A ‘clone’ is a population of cells derived from a single cell or common ancestor by mitosis.

A ‘cell line’ is a clone of a primary cell that is capable of stable growth in vitro for many generations. In some examples provided herein, cells are transformed by transfecting the cells with DNA.

The terms ‘express’ and ‘produce’ are used synonymously herein and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term ‘subject’ refers to human and non-human animals, including all vertebrates, e.g., mammals and non-mammals, such as non-human primates, mice, rabbits, sheep, goats, dogs, cats, horses, cows, chickens, amphibians, and reptiles. In most particular embodiments of the described methods, the subject is a human.

The terms ‘treating’ or ‘treatment’ refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

### Sequence Homology

When referring to CLL1, it will be appreciated that reference thereto encompasses fragments thereof, as well as related polypeptides, which include, but are not limited to, allelic variants, splice variants, derivative variants, substitution variants, deletion variants, and/or insertion variants including the addition of an N-terminal methionine, fusion polypeptides, and interspecies homologs. In certain embodiments, a CLL1 polypeptide includes terminal residues, such as, but not limited to, leader sequence residues, targeting residues, amino terminal methionine residues, lysine residues, tag residues and/or fusion protein residues.

Amino acid sequence of an example isoform of CLL1 to which the antibodies of the present invention bind is provided as SEQ ID NO: 1.

The DNA sequence of CLL1 (SEQ ID NO: 1) is provided SEQ ID NO: 2. Splice variants and other isoforms of CLL1 are known and are encompassed by the present disclosure,

For all aspects and embodiments of the present invention, the amino acid sequence of CLL1 encompasses a polypeptide that has at least 50%, 60%, 70%, 80%, 90%, 95% or 99% complete sequence identity to CLL1 (for example SEQ ID NO: 1). Likewise, the CLL1 polynucleotide sequence can comprise a polynucleotide that has at least 50%, 60%, 70%, 80%, 90%, 95% or 99% complete sequence identity to CLL1 (for example, SEQ ID NO: 2). Sequence identity can also be to a fragment or portion of the full length polynucleotide or polypeptide. Hence, a sequence may have only 50% overall sequence identity with a sequence of the invention but in a particular region, domain or subunit could share 80%, 90%, or as much as 99% sequence identity with sequence of the invention.

According to the present invention, homology to the nucleic acid sequence of SEQ ID NOs: 3 to 92 is not limited simply to sequence identity. Many nucleic acid sequences can demonstrate biologically significant homology to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY).

The present application is the first to show a systematic high-throughout screen output of a CAR-T cell library. This allowed identification of multiple novel antibody or antigen binding fragments (e.g. scFvs) and their associated light chain and heavy chain CDRs, that bind to CLL1, and outperform other known antibody or antigen binding fragments in several experimental models.

Accordingly, in at least one aspect, the invention provides an antibody or antigen binding fragments having specificity for CLL1 (Antibody Nos. 1 to 10), wherein the antibody or antigen binding fragments have an antigen binding domain comprising the amino acid sequences (SEQ ID NOS: 3 to 92) as shown in Table 1 below:

TABLE 1
Exemplified antibodies or antigen binding fragments of the present invention

| Antibody | | | V_{H} | | | | V_{L} | | |
|---|---|---|---|---|---|---|---|---|---|
| No. | scFv. | V_{H} | CDR1 | CDR2 | CDR3 | V_{L} | CDR1 | CDR2 | CDR3 |
| 1 | SEQ ID | SEQ ID | SEQ ID | SEQ ID | SEQ ID | SEQ ID | SEQ ID | SEQ ID | SEQ ID |
| | NO. 3 | NO. 13 | NO. 23: | NO. 33: | NO. 43: | NO. 53 | NO. 63: | NO. 73: | NO. 83: |
| | | | SYAMD | AISPSGG | REYWG | | RASQD | GASNLQ | QQAYG |
| | | | | GTVYNEK | ELFDL | | IGNWL | S | YPFT |
| | | | | FQG | | | N | | |
| 2 | SEQ ID | SEQ ID | SEQ ID | SEQ ID | SEQ ID | SEQ ID | SEQ ID | SEQ ID | SEQ ID |
| | NO. 4 | NO. 14 | NO. 24: | NO. 34: | NO. 44: | NO. 54 | NO. 64: | NO. 74: | NO. 84: |
| | | | SYSMS | VISSGTG | KNSWG | | QASQN | DASNLA | QQSYNT |
| | | | | STYYNEK | DDFDL | | INNFLA | T | PLT |
| | | | | FQG | | | | | |
| 3 | SEQ ID | SEQ ID | SEQ ID | SEQ ID | SEQ ID | SEQ ID | SEQ ID | SEQ ID | SEQ ID |
| | NO. 5 | NO. 15 | NO. 25: | NO. 35: | NO. 45: | NO. 55 | NO. 65: | NO. 75: | NO. 85: |
| | | | SYDID | GISPQSG | RVHGD | | QASQD | HASSLE | QQYYN |
| | | | | GTSYNEK | YHFDY | | VSTDL | S | YPLT |
| | | | | FQG | | | A | | |
| 4 | SEQ ID | SEQ ID | SEQ ID | SEQ ID | SEQ ID | SEQ ID | SEQ ID | SEQ ID | SEQ ID |
| | NO. 6 | NO. 16 | NO. 26: | NO. 36: | NO. 46: | NO. 56 | NO. 66: | NO. 76: | NO. 86: |
| | | | SYDMS | AINPSGD | GVGW | | QASQD | SASSLQ | QQSNYL |
| | | | | FTSYNEK | SFLDY | | VRTWL | T | PFT |
| | | | | FQG | | | A | | |
| 5 | SEQ ID | SEQ ID | SEQ ID | SEQ ID | SEQ ID | SEQ ID | SEQ ID | SEQ ID | SEQ ID |
| | NO. 7 | NO. 17 | NO. 27: | NO. 37: | NO. 47: | NO. 57 | NO. 67: | NO. 77: | NO. 87: |
| | | | DYAMD | WISGQS | KWLDG | | KASEDI | SASSLQ | QQAYYL |
| | | | | GSTSYNE | PWFDV | | HSDLA | S | PLT |
| | | | | KFQG | | | | | |
| 6 | SEQ ID | SEQ ID | SEQ ID | SEQ ID | SEQ ID | SEQ ID | SEQ ID | SEQ ID | SEQ ID |
| | NO. 8 | NO. 18 | NO. 28: | NO. 38: | NO. 48: | NO. 58 | NO. 68: | NO. 78: | NO. 88: |
| | | | DYGM | YISGGTG | RYGAN | | RASQD | GASRLY | QQGYN |
| | | | D | RTAYNEK | DGFDI | | ISSYLN | T | TPYT |
| | | | | FQG | | | | | |
| 7 | SEQ ID | SEQ ID | SEQ ID | SEQ ID | SEQ ID | SEQ ID | SEQ ID | SEQ ID | SEQ ID |
| | NO. 9 | NO. 19 | NO. 29: | NO. 39: | NO. 49: | NO. 59 | NO. 69: | NO. 79: | NO. 89: |
| | | | HYAMD | GISPQSG | RVHGD | | QASQD | HASSLE | QQYYN |
| | | | | GTSYNEK | YHFDY | | VSTDL | S | YPLT |
| | | | | FQG | | | A | | |
| 8 | SEQ ID | SEQ ID | SEQ ID | SEQ ID | SEQ ID | SEQ ID | SEQ ID | SEQ ID | SEQ ID |
| | NO. 10 | NO. 20 | NO. 30: | NO. 40: | NO. 50: | NO. 60 | NO. 70: | NO. 80: | NO. 90: |
| | | | SYAMS | AINSQGK | RPDHG | | RASQG | GASSLQ | QQYSSF |
| | | | | STYYADS | DFMDV | | ISNWL | S | PTT |
| | | | | VKG | | | A | | |
| 9 | SEQ ID | SEQ ID | SEQ ID | SEQ ID | SEQ ID | SEQ ID | SEQ ID | SEQ ID | SEQ ID |
| | NO. 11 | NO. 21 | NO. 31: | NO. 41: | NO. 51: | NO. 61 | NO. 71: | NO. 81: | NO. 91: |
| | | | DYDMS | VISSSGG | RSPMN | | KASQG | VASRLE | QQSSFL |
| | | | | STTYNEK | EDWFD | | VSTWL | S | PIT |
| | | | | FQG | Y | | N | | |
| 10 | SEQ ID | SEQ ID | SEQ ID | SEQ ID | SEQ ID | SEQ ID | SEQ ID | SEQ ID | SEQ ID |
| | NO. 12 | NO. 22 | NO. 32: | NO. 42: | NO. 52: | NO. 62 | NO. 72: | NO. 82: | NO. 92: |
| | | | HYAMS | GIYGGGD | KASWE | | KASQDI | GASSLA | QQSYNY |
| | | | | STNYNEK | DTFDY | | RNYLA | S | PLT |
| | | | | FQG | | | | | |

The antibodies, or antigen binding fragments, of the present invention were identified from a functional high-throughput screen of CAR-T cells, wherein the CAR-T cells express a unique chimeric antigen receptor (CARs), each CAR comprising an antibody or antigen binding fragment and/or scFv derived from a diverse scFv library. The antibodies or antigen binding fragments as identified in Table 1 are those exhibiting surprisingly good functional response (i.e. were positive hits) in various assays against cells expressing CLL1 and are therefore deemed to be superior CAR-T cells over that known in the art and provide novel and effective antibodies or antigen binding fragments of CLL1.

The high-throughput screening method employed to identify CLL1 antibodies or antigen binding fragments, and/or bi-specific, tri-specific or multi-specific antigen binding molecules and/or antibody-drug conjugates and/or CAR-T cells derived therefrom is described in the Applicant's co-pending International application PCT/GB2022/050158 (published as WO2022/157500), the content of which is incorporated by reference.

In embodiments, the antibody or antigen binding fragment can be an isolated antibody having specificity for CLL1 (suitably human CLL1) and can be a full-length antibody or an antibody fragment. The antibody can be polyclonal, monoclonal, recombinant, chimeric, or humanised. Furthermore, the antibody can be of any isotype including without limitation IgA, IgD, IgE, IgG, or IgM. Thus, for example, the antibody can be any IgA such as IgA1 or IgA2, or any IgG such as IgG1, IgG2, IgG3, IgG4, or synthetic IgG. The antibody can also be any antibody fragment having specificity for CLL1, such as F(ab)₂, Fv, scFv, F(ab′)₂, F(ab), V_{L}, V_{H}, dsFv, Fv, scFv-Fc, (scFv)₂, a diabody, and a bivalent antibody. The antibody can be any modified or synthetic antibody, including, but not limited to, non-depleting IgG antibodies, T-bodies, or other Fe or Fab variants of antibodies.

In some embodiments, the invention provides an antibody or antigen binding fragment with avidity for CLL1 of about 10 UM or less, 5 UM or less, 2 UM or less, 1 UM or less, 500 nM or less, 400 nM or less, 300 nM or less, or 200 nM or less, or 100 nM or less, or 75 nM or less, or 50 nM or less, or 25 nM or less, or 10 nM or less, or 5 nM or less. Avidity can be measured using art-known techniques, such as ELISA or BIACORE.

The antibody or antigen binding fragment of the invention can be produced by any suitable technique, for example, using any suitable eukaryotic or non-eukaryotic expression system. In certain embodiments, the antibody is produced using a mammalian expression system. Alternatively, the antibody or antigen binding fragment of the invention can be produced using a suitable non-eukaryotic expression system such as a bacterial expression system. Bacterial expression systems can be used to produce fragments such as a F(ab)₂, Fv, scFv, F(ab′)₂, F(ab), V_{L}, V_{H}, dsFv, Fv, scFv-Fc, (scFv)₂, and diabodies.

The antibody or antigen binding fragment of the invention can be conjugated to a synthetic molecule. Conjugation of the antibody or antigen binding fragment of the invention to the synthetic molecule may be by any suitable method, for example recombinant engineering. The synthetic molecule can be any molecule such as a drug targeting a tumour or tumour cells. The synthetic molecule can also be a peptide/protein or an antibody or an antigen binding fragment such that the resultant molecule has specificity for more than one antigen, wherein the resulting fusion protein can be produced by conventional recombinant protein expression systems and methods.

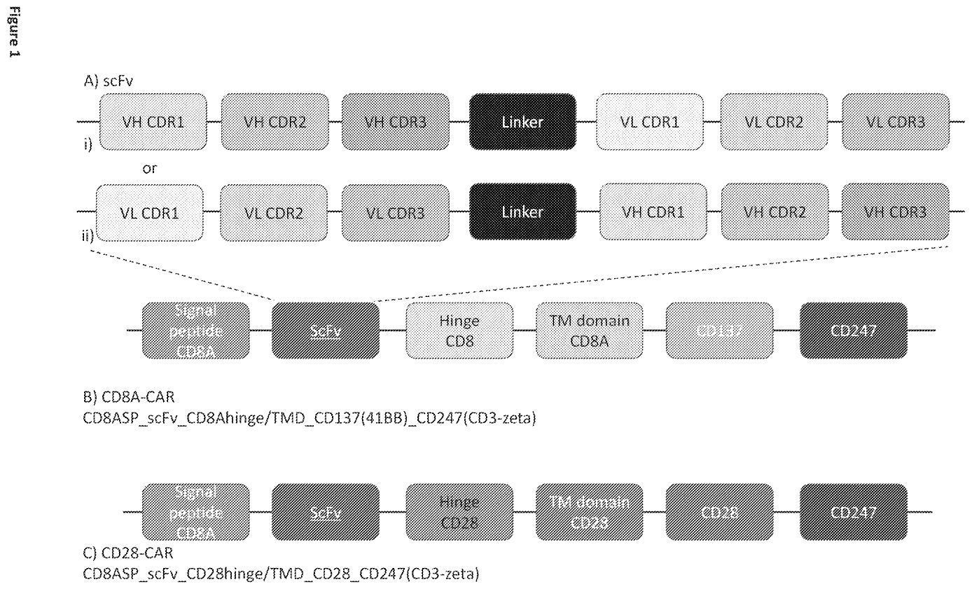

In this respect, a further aspect of the invention relates to chimeric antigen receptors (CARs). CARS, are engineered receptors, which confer specificity for a desired antigen onto an immune effector cell, such as a T-cell. CARs may be expressed on the extracellular surface of a cell. Expression may be by any suitable means via retroviral vector expression. The most common form of these molecules are fusions of an antibody or antigen binding fragment, such as an scFv, to the transmembrane and intracellular domains of a native T-cell activation receptor, such as CD3, typically CD3-zeta. Such molecules result in the transmission of a signal, suitable an activation signal, in response to recognition by the scFv of its target. ‘First-generation’ CARs typically have the intracellular domain from the CD3-zeta chain, which is the primary transmitter of signals from endogenous TCRs. ‘Second-generation’ CARs add intracellular signaling domains from various costimulatory protein receptors (e.g., CD28, CD137, 41BB, ICOS) to the intracellular portion of the CAR to provide additional signals to the T cell (see FIG. 1). Preclinical studies have indicated that the second generation of CAR designs improves the antitumor activity of T cells (Maher, et al. (2002) Nat. Biotechnol. 20:70-75; Kowolik, et al. (2006) Cancer Res. 66:10995-11004). ‘Third-generation’ CARs combine multiple signaling domains, such as CD3z-CD28-41BB or CD3z-CD28-OX40, to further augment potency (Zhao, et al. (2009) J. Immunol. 183:5563-5574; Pule, et al. (2005) Mol. Ther. 12:933-941; Zhong, et al. (2010) Mol. Ther. 18:413-420). Accordingly, in an embodiment of this invention, a CAR is provided that comprises an anti-CLL1 antibody or antigen binding fragment, such as an scFv fragment, as defined in SEQ ID NOS 3 to 12.

CARs of this invention can be prepared using standard recombinant protein techniques using sequences of CD3, e.g. CD247 (CD3-zeta) and optionally other costimulatory molecules known in the art. For example, the human CD247 (CD3-zeta) sequence is available under GENBANK accession number NP_932170, the human CD28 sequence is available under GENBANK accession number NP 006130, the human CD8A sequence is available under GENBANK accession number AAH25715, and the human CD137 sequence is available under GENBANK accession number NP_001552.

In particular embodiments, the CAR of the present invention are represented by the SEQ ID NOS: 93 to 112. These CARs include, but not necessarily limited to, either:
1) a human CD247 (CD3-zeta) cytoplasmic domain (SEQ ID NO:113), a human CD137 co-stimulatory domain (SEQ ID NO:114); a human CD8A transmembrane domain (SEQ ID NO: 115), a human CD8A hinge domain (SEQ ID NO:116); an antigen binding fragment of the present invention (SEQ ID NOS: 3 to 12) optionally comprising a linker (SEQ ID NO: 117 or 118) between the V_{H} and V_{L} sequences; and a CD8A extracellular signal peptide (SEQ ID NO: 119); or
2) a human CD247 (CD3-zeta) cytoplasmic domain (SEQ ID NO:113), a human CD28 co-stimulatory domain (SEQ ID NO:120); a human CD28 transmembrane domain (SEQ ID NO: 121), a human CD28 hinge domain (SEQ ID NO:122); an antigen binding fragment of the present invention (SEQ ID NOS: 3 to 12) optionally comprising a linker (SEQ ID NO: 117 or 118) between the V_{H} and V_{L} sequences; and an CD8A extracellular signal peptide (SEQ ID NO: 119).

In specific embodiments, the CARs of the present invention have sequences defined in SEQ ID NOS: 93 to 102.

In some embodiments, the antibody or antigen binding fragment can be conjugated to a synthetic molecule that can confer specificity for one or more antigens in addition to CLL1. For example, the antibody of the invention can be engineered (e.g. as a bivalent diabody or a conjugated Fab dimer or trimer) to have specificity for CLL1 and another tumour antigen, e.g., an antigen associated with a disease or disorder as disclosed herein. Alternatively, the antibody can be engineered to have specificity for CLL1 and an antigen that promotes activation or targeting of other cells, such as cytotoxic effector cells or T cells. Accordingly, the invention also includes bispecific, tri-specific and multi-specific molecules such as BiTEs (bi-specific T-cell engagers) and DART™s (dual affinity retargeting reagents).

As is known in the art, a bi-specific T-cell engager (BiTE) refers to a single polypeptide chain molecule having two antigen binding domains, one of which binds to a T-cell antigen (e.g., CD3) and the second of which binds to an antigen present on the surface of a target cell (WO 05/061547; Baeuerle, et al. (2008) Drugs of the Future 33:137-147; Bargou, et al. (2008) Science 321:974-977). BiTE antibodies have been constructed to various target antigens including CD19, EpCAM, Her2/neu, EGFR, CD66e (or CEA, CEACAM5), CD33, EphA2, and MCSP (or HMW-MAA) (Baeuerle, et al. (2009) Curr. Opin. Mol. Ther. 11:22-30). Accordingly, in another embodiment of this invention, an anti-CLL1 antibody or antigen binding fragment (e.g. a scFv) is a component of a bi-specific T-cell engager (BiTE). In particular embodiments, the bi-specific T-cell engager (BiTE) of this invention is composed of an anti-CLL1 antibody or antigen binding fragment and an anti-CD3 antibody fragment fused together by a linker, e.g., a flexible protein linker. See, for example, U.S. Pat. No. 5,929,212. The term ‘BiTE’ as used herein may encompass but is not limited to the bispecific T-cell engager named BiTE® available from Amgen®.

In embodiments, the antibody fragment that specifically binds CD3 comprises:
(a) a heavy chain variable region comprising,
   (i) a CDR1 of SEQ ID NO:123,
   (ii) a CDR2 of SEQ ID NO:124, and
   (iii) a CDR3 of SEQ ID NO:125; and
(b) a light chain variable region comprising,
   (i) a CDR1 of SEQ ID NO:126,
   (ii) a CDR2 of SEQ ID NO:127, and
   (iii) a CDR3 of SEQ ID NO:128.

In other embodiments, the invention provides the antibody or antigen binding fragment of the invention coupled to a synthetic molecule as defined herein to provide a DART™. DART™ refers to an immunoglobulin molecule that includes at least two polypeptide chains that associate (especially through a covalent interaction) to form at least two antigen binding sites, which may recognize the same or different antigens. Each of the polypeptide chains of a DART™ include an immunoglobulin light chain variable region and an immunoglobulin heavy chain variable region, but these regions do not interact to form an epitope binding site. Rather, the immunoglobulin heavy chain variable region of one (e.g., the first) of the DART™ polypeptide chains interacts with the immunoglobulin light chain variable region of a different (e.g. the second) DART™ polypeptide chain to form an epitope binding site. Similarly, the immunoglobulin light chain variable region of one (e.g. the first) of the DART™ polypeptide chains interacts with the immunoglobulin heavy chain variable region of a different (e.g., the second) DART™ polypeptide chain to form an epitope binding site. DART™s may be monospecific, bi-specific, tri-specific, etc., thus being able to simultaneously bind one, two, three or more different antigens (which may be of the same or of different antigens). DART™Ms may additionally be monovalent, bivalent, trivalent, tetravalent, pentavalent, hexavelent, etc., thus being able to simultaneously bind one, two, three, four, five, six or more molecules. These two attributes of DART™Ms (i.e., degree of specificity and valency may be combined, for example to produce bispecific antibodies (i.e., capable of binding two epitopes) that are tetravalent (i.e. capable of binding four sets of antigens/epitopes), etc. The construction of DART™ molecules is disclosed in WO 2006/113665, WO 2008/157379, and WO 2010/080538. Accordingly, in another embodiment of this invention, an anti-CLL1 antibody or antigen binding fragment is included in a DART™.

In other embodiments, the antibody or antigen binding fragment of the present invention is conjugated, linked or joined to other synthetic molecules including therapeutic agents (or ‘payloads’) such as cytotoxic, cytostatic, or anti-angiogenic agents and radioisotopes, labels or nanoparticles. Such molecules are terms antibody-drug conjugates or ADCs. Examples of ADCs include: Gemtuzumab ozogamicin, Brentuximab vedotin, Trastuzumab emtansine, Trastuzumab emtansine and Trastuzumab emtansine.

The invention provides a method of inhibiting cells that express CLL1 (CLL1 cells) by contacting the cells with an antibody, antigen binding fragment or antibody conjugated to a synthetic molecule (e.g. a bispecific T-cell engager such as a BiTE, DART™, or ADC) of the invention. The method can be used to inhibit CLL1 cells in vitro or in a subject (i.e., in vivo). The contacted CLL1 cells can be in, for example, a cell culture or animal model of a disorder associated with aberrant expression or levels of CLL1. The method is useful, for example, to measure and/or rank (relative to another antibody) the antibody's inhibitory activity for a CLL1 cell type. Inhibiting CLL1 cells can include blocking or reducing the activity or growth of CLL1 cells. Inhibiting can also include the killing of CLL1 cells. Cytotoxicity of an antibody, antibody fragment or antibody conjugated to a synthetic molecule such as a BITE, DART™, or ADC of the invention can be assessed using any conventional assay including, e.g. a lactate dehydrogenase cytotoxicity assay such as the CYTOTOX 96 non-radioactive cytotoxicity assay commercially available from PROMEGA. Similarly, the invention provides an antibody, antigen binding fragment or antibody conjugated to a synthetic molecule (e.g. a bispecific T-cell engager such as a BiTE, DART™, or ADC) of the invention for use in inhibiting and killing cells expressing CLL1.

The invention also provides a method of treating a subject that has, is suspected to have, or is at risk for a disorder associated with aberrant levels of CLL1 or CLL1 cells. As used in the context of the present invention, the term ‘aberrant’ is intended to include increased or decreased CLL1 expression on a cell as compared to expression of CLL1 in normal or healthy cells, or an increase in cells expressing CLL1. Generally, the method of treatment includes administering a therapeutically effective amount of an isolated antibody, antibody fragment or fusion protein of the invention to the subject. The antibody can be any anti-CLL1 antibody, antigen binding fragment (e.g. chimeric, humanised, synthetic, F(ab)2, Fv, scFv, F(ab′)₂, F(ab), V_{L}, V_{H}, dsFv, Fv, or (scFv)₂) or antibody conjugated to a synthetic molecule (e.g., a bispecific T-cell engager such as a BITE, DART™, or ADC) as described herein. Similarly, the invention provides an antibody, antigen binding fragment or antibody conjugated to a synthetic molecule (e.g., a bispecific T-cell engager such as a BiTE, DART™, or ADC) of the invention for use in medicine, and for the treatment of disease and disorders associated with aberrant or elevated CLL1 expression.

Disorders that can be treated include, for example, cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

The invention also provides a method of treating a subject that has, is suspected to have, or is at risk for a disorder associated with elevated levels of CLL1 on a cell, or by elevated levels of CLL1-cells by adoptive transfer of the recombinant host cells, e.g. T-cells described herein, which express an antibody or antigen binding fragment conjugated to a synthetic molecule of the invention as a CAR that selectively binds CLL1. Similarly, the invention provides recombinant cells expressing an antibody or antigen binding fragment conjugated to a synthetic molecule (e.g. CAR) of the invention for use in medicine.

Recombinant technology can be used to introduce CAR-encoding genetic material into any suitable T-cells, e.g. effector memory T-cells from the subject to be treated. The recombinant T-cells are transferred, typically by infusion, to the patient. The transferred T-cells of the invention can then mount an immune response against CLL1 expressing cells (CLL1-cells) in the patient.

The adoptive transfer method can be used, for example, to treat subjects that have or are suspected to have cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

In embodiments, and suitable for use in treatment, the invention also provides a pharmaceutical composition containing an antibody or antigen binding fragment or antibody conjugated to a synthetic molecule (e.g., a CAR, a bispecific T-cell engager such as a BITE, DART™, or ADC) as described herein and a pharmaceutically acceptable carrier. Pharmaceutical compositions can be prepared from any of the antibody, antigen binding fragment (e.g. chimeric, humanised, synthetic, F(ab)2, Fv, scFv, F(ab′)₂, F(ab), V_{L}, V_{H}, dsFv, Fv, or (scFv)₂) or antibody conjugated to a synthetic molecule (e.g., a CAR, a bispecific T-cell engager such as a BITE, DART™, or ADC) as described herein.

The composition of the invention can include a carrier for the antibody or antigen binding fragment, or antibody conjugated to a synthetic molecule, desirably a pharmaceutically acceptable carrier. The pharmaceutically acceptable carrier can be any suitable pharmaceutically acceptable carrier. The term ‘pharmaceutically acceptable carrier’, as used herein, means one or more compatible solid or liquid fillers, diluents, other excipients, or encapsulating substances, which are suitable for administration into a human or veterinary patient (e.g. a physiologically acceptable carrier or a pharmacologically acceptable carrier). The term ‘carrier’ denotes an organic or inorganic ingredient, natural or synthetic, with which the active ingredient is combined to facilitate the application. The pharmaceutically acceptable carrier can be co-mingled with one or more of the active components, e.g., a hybrid molecule, and with each other, when more than one pharmaceutically acceptable carrier is present in the composition in a manner so as not to substantially impair the desired pharmaceutical efficacy. ‘Pharmaceutically acceptable’ materials typically are capable of administration to a patient without the production of significant undesirable physiological effects such as nausea, dizziness, rash, or gastric upset.

The pharmaceutical composition can contain suitable buffering agents, including, for example, acetic acid in a salt, citric acid in a salt, boric acid in a salt, and phosphoric acid in a salt. The pharmaceutical compositions also optionally can contain suitable preservatives, such as benzalkonium chloride, chlorobutanol, parabens, and thimerosal.

The pharmaceutical composition can be presented in unit dosage form and can be prepared by any suitable method, many of which are well-known in the pharmaceutical arts. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

### High Throughput Screening to Identify CARs/Antibodies

The present invention benefits from the high-throughout screening methodology as described in the co-pending International application no. PCT/GB2022/050158, the content of which is incorporated herein by reference.

The high-throughout screening method allows resources to be focused primarily on the diversity of the CAR library and identification of the CARs that are able to demonstrate promising activity in a functional assay relevant to a clinical context.

The invention is described in greater detail by the following non-limiting examples.

### EXAMPLES

### Example 1

A synthetic human scFv phage library was panned against recombinant human CLL1 protein (R&D Biosystems™). To this end, the library was grown to log phase, and then rescued with M13KO7 helper phage (Antibody Design Lab™, PH010L) before being amplified overnight at 32° C. in a shaker. The phage library was subsequently precipitated with PEG/NaCl, re-suspended in PBS and stored at −80° C. Protein G coated magnetic beads were coated with hCLL1-Fc or Fc recombinant protein in PBS and subsequently blocked in PBS+ BSA. Phage particles were incubated for 30 minutes with negative magnetic particles. Subsequently, magnetic particles were pelleted and the supernatant was incubated for 1 h with hCLL1 coated magnetic beads under rotation. After 1 h incubation, unbound and non-specifically bound phages were washed away by rinsing the beads with PBST. Bound phages were eluted by 100 mM triethylamine (TEA), and the eluate was neutralized by 1 M Tris-HCl (pH 7.4). The eluate was then used to infect exponentially growing E. coli TGI cells. The panning was repeated for an additional two to four cycles.

### Example 2

After the last panning step, scFv sequences were PCR amplified from the eluted phages or the isolated plasmids using Q5 DNA polymerase (NEB) and scFv-specific forward and reverse primers. Resulting amplicon was cleaned by PCR clean-up and 1 μg PCR product were digested with restriction enzymes for 2 h at 37° C. In parallel, a CAR scaffold library, consisting of pooled plasmids containing different CAR scaffolds with variation in hinge domain, transmembrane domain and intracellular signalling domains, was digested using Esp3I. After 2 h incubation the CAR scaffold library and the scFv amplicons were cleaned by PCR clean-up columns and ligated using T4 DNA ligase (Thermo Fisher™) for 12 h over-night. The resulting CAR-library was electroporated into electrocompetent bacteria and grown overnight at 30° C., followed by plasmid maxi-prep isolation. The CAR sequences from the resulting plasmid library were briefly PCR amplified and sequenced on an Oxford Nanopore Technology™ MinION™, using the amplicon sequencing kit.

### Example 3

In order to assess if the CAR library was functionally expressed and recognizing CLL1, the CAR library against CLL1 was used to produce lentiviral vector particles. The lentiviral library was titrated and MOI (Multiplicity of Infection) determined on Jurkat cell line. Subsequently, the library was transduced into human T cells or Jurkat cell line, to reach a transduction efficiency of around 20%. The resulting CAR-T cell library was expanded for six more days after transduction and assessed for CLL1 binding. CLL1-CAR-T cell library cells were stained with CLL1-Fc fusion protein or a negative control protein (recombinant Fc, R&D systems). In a second step, cells were stained with PE-conjugated anti-Fc and APC-conjugated anti-CD34 antibody to detect transduced cells. The expression of CARs on the cell surface was assessed.

### Example 4

A CAR-T cell library was produced by activation of Peripheral Blood Mononuclear Cells (PBMCs) with TransAct™ (Miltenyi™) in the presence of human IL-2 (100 IU/ml) and transduced at day two with the lentiviral CLL1 CAR library. The next day, cells were washed and further expanded until day 8 of the process. Transduction was assessed by flow cytometry and transduced cells were enriched through CD34 microbeads (Miltenyi™). Purity was assessed by flow cytometry. The CAR-T-cell library was co-cultured with CLL1-positive cells (HL60) or no cells. After 6 h activation, CAR-T cells were prepared for single cell sequencing analysis. CAR-T cell libraries from different donors were prepared for 10×genomics single cell gene expression analysis (10× genomics 3′ sequencing kit V3 or Single Cell 5′ Kit v2) and sequenced on a NovaSeq™ 6000 (Illumina™). Cell ranger™ software (10× Genomics™) was used for downstream processing and alignment of reads. In order to deconvolute the single cell sequencing data and identify the CAR sequence and cell barcode identity, CAR sequences were amplified using long PCR with a forward read 1 primer and CAR-specific reverse primer. After 10 cycles of PCR, the product was cleaned using SPRI bead clean-up and a second, nested PCR was performed. PCR products were barcoded and an Oxford Nanopore library was prepared using the Oxford Nanopore ligation sequencing kit. Libraries were then sequenced on a MinION® flow cell and CAR sequences and 10× cell barcodes were determined.

### Example 5

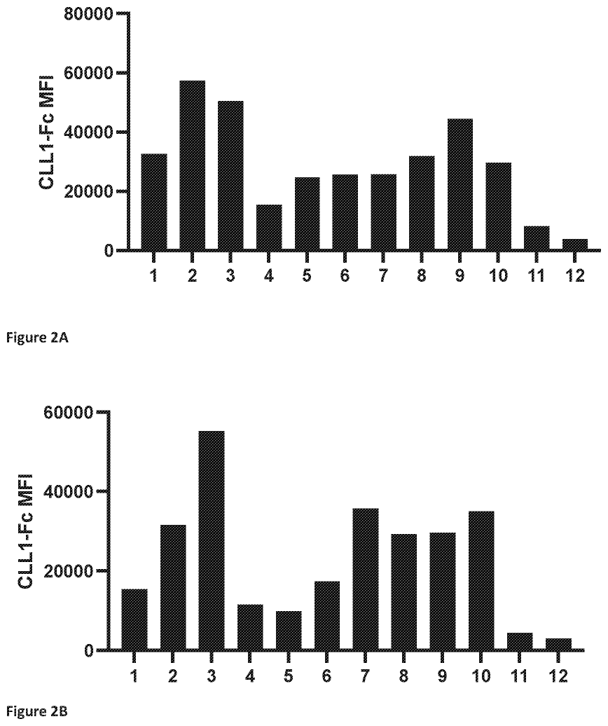

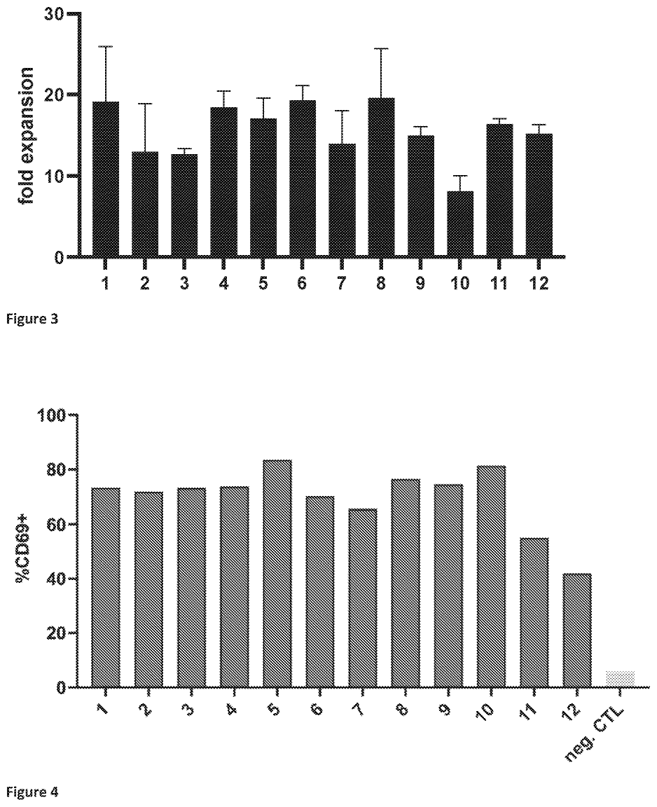

Single CAR constructs were selected based on best activation propensity from the single cell gene expression analysis, and CAR sequences were synthetized by Integrated DNA technologies (IDT). CAR sequences were cloned into a lentiviral expression construct, encoding a 2A peptide and a truncated CD34 reporter. Lentiviral vectors were produced with the individual CAR constructs and titrated on Jurkat cell line. PBCMs were activated on day 0 with TransAct™ (Miltenyi™) and transduced with equal MOI (Multiplicity of Infection) of lentiviral vector two days after activation, in the presence of recombinant human IL2 (Miltenyi™). On day 4, cells were washed, counted and seeded for expansion until day 8. Cell expansion was quantified using LUNA-FL cell counter and live/dead fluorescence dye. Average cell expansion between donors was calculated as the fold change of total cell count between day 4 and day 8 (FIG. 3). Cells were subsequently used for functional assays. The affinity/avidity with which the different CARs bind to CLL1 was assessed. Cells transduced with the different CLL1-CAR constructs were stained with CLL1-Fc fusion protein (R&D systems). In a second step, cells were stained with PE-conjugated anti-Fc and an APC-conjugated anti-CD34 antibodies. Median fluorescence intensity of CLL1-Fc staining is shown for CD34+ CAR-T cells (FIGS. 2A and 2B).

The labels on the x axis indicate different CAR constructs, as indicated below.

| Label | CLL1-CAR construct |
|---|---|
| 1 | CD8A-CAR construct comprising Antibody 1 (SEQ ID No: 93) |
| 2 | CD8A-CAR construct comprising Antibody 2 (SEQ ID No: 94) |
| 3 | CD8A-CAR construct comprising Antibody 3 (SEQ ID No: 95) |
| 4 | CD8A-CAR construct comprising Antibody 4 (SEQ ID No: 96) |
| 5 | CD8A-CAR construct comprising Antibody 5 (SEQ ID No: 97) |
| 6 | CD8A-CAR construct comprising Antibody 6 (SEQ ID No: 98) |
| 7 | CD8A-CAR construct comprising Antibody 7 (SEQ ID No: 99) |
| 8 | CD8A-CAR construct comprising Antibody 8 (SEQ ID No: 100) |
| 9 | CD8A-CAR construct comprising Antibody 9 (SEQ ID No: 101) |
| 10 | CD8A-CAR construct comprising Antibody 10 (SEQ ID No: 102) |
| 11 | Comparative CLL1-CAR construct from US2020/0115457A1 (SEQ ID No: 129) |
| 12 | Comparative CLL1-CAR construct from US2013/0295118A1 (SEQ ID No: 102) |

CAR 11 and CAR 12 comprise respective antibody/antigen-binding fragments sequences defined as Antibody No. 11 and Antibody No. 12 (Table 2).

TABLE 2
Comparative example antibodies from US2020/0115457A1 and US2013/0295118A1 respectively

| Antibody | | | V_{H} | | | | V_{L} | | |
|---|---|---|---|---|---|---|---|---|---|
| No. | scFv. | V_{H} | CDR1 | CDR2 | CDR3 | V_{L} | CDR1 | CDR2 | CDR3 |
| 11 | SEQ ID | SEQ ID | SEQ ID | SEQ ID | SEQ ID | SEQ ID | SEQ ID | SEQ ID | SEQ ID |
| | NO. 133 | NO. 135 | NO. 137: | NO. 139: | NO. 141: | NO. 143 | NO. 145: | NO. 147: | NO. 149 |
| | | | SYYWS | YIYYSGSTN | LVYCGGD | | QASQDI | DASNLET | QQYGNLP |
| | | | | YNPSLKS | CYSGFDY | | NNFLN | | FT |
| 12 | SEQ ID | SEQ ID | SEQ ID | SEQ ID | SEQ ID | SEQ ID | SEQ ID | SEQ ID | SEQ ID |
| | NO. 134 | NO. 136 | NO. 138: | NO. 140: | NO. 142: | NO. 144 | NO. 146: | NO. 148: | NO. 150: |
| | | | SYFIH | FINPYNDGS | DDGYYGY | | RATQELS | AASTLDS | LQYAIYPY |
| | | | | KYNEKFKG | AMDY | | GYLS | | T |

As is evident from FIGS. 2A and 2B, each of the CARs corresponding to CAR SEQ ID NOs 93 to 102, comprising the scFv SEQ ID NOs 3 to 12 (and the associated CDRs as detailed in Table 1) showed enhanced activity against CLL1+ cells than the prior art CARs. This demonstrates the surprisingly beneficial effect of the antibody/antigen binding fragments of the present invention, and the utility of such antibody/antigen binding fragments in CARs, other bi-specific, tri-specific and multi-specific molecules (such as BiTEs and DART™s), and ADCs.

### Example 6

To further evaluate the activity of different anti-CLL1 CARS, CLL1-CAR T-cells were evaluated for cellular toxicity and activity against cancer cells expressing CLL1, or controls lacking expression of CLL1.

The ability of the different CARs to elicit functional responses was explored by incubating the CLL1-CAR T-cells for 24 hours at 1:1 ratio with CLL1-expressing cancer cell line (HL60). After 24 h co-culture, cells were stained with anti-CD3 Pacific Blue and anti-CLL1 APC in order to separate T cells and cancer cells. Cells were acquired and counted using flow cytometry and cell killing was calculated compared to HL60 cells cultured in the absence of CAR-T cells (FIG. 5).

CAR-T cell activation was assessed via CD69 upregulation on CD3+, CD34+ CAR-T cells as an indicator for T-cell activation (FIG. 4).

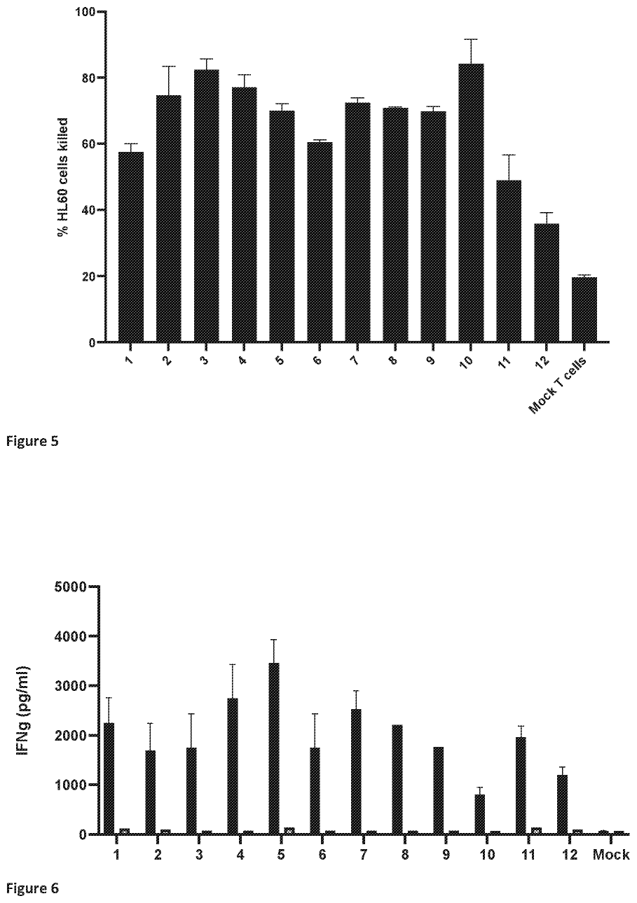

CAR-T potency was assessed by collecting supernatant of CAR-T cells stimulated with HL60 cells or non-stimulated controls. After 24 h of co-culture supernatant was collected and assessed for cytokine production via IFNg ELISA (FIG. 6). Left bar on each CAR-T number indicate IFNg values of CAR-T cells stimulated with HL60 cells. Right bar indicate non-stimulated controls.

As is evident from FIG. 4, each of the CARs corresponding to CAR SEQ ID NOs 93 to 102, comprising the scFvs SEQ ID NOs 3 to 12 (and the associated CDRs as detailed in Table 1) showed enhanced activity via CD69 upregulation against CLL1+ cells than the prior art CARs. This demonstrates the surprisingly beneficial effect of the antibody/antigen binding fragments of the present invention, and the utility of such antibody/antigen binding fragments in CARs, other bi-specific, tri-specific and multi-specific molecules (such as BiTEs and DART™s), and ADCs.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

Sequence listing:

| SEQ ID | | |
|---|---|---|
| NO: | Description | Sequence |
| 1 | CLL1 amino acid | MSEEVTYADLQFQNSSEMEKIPEIGKFGEKAPPAPSHVWRPAALFLTLLCLL |
| | | LLIGLGVLASMFHVTLKIEMKKMNKLQNISEELQRNISLQLMSNMNISNKIRN |
| | | LSTTLQTIATKLCRELYSKEQEHKCKPCPRRWIWHKDSCYFLSDDVQTWQE |
| | | SKMACAAQNASLLKINNKNALEFIKSQSRSYDYWLGLSPEEDSTRGMRVDN |
| | | IINSSAWVIRNAPDLNNMYCGYINRLYVQYYHCTYKKRMICEKMANPVQLG |
| | | STYFREA |
| 2 | CLL 1 nucleic acid | atgtctgaagaagttacttatgcagatcttcaattccagaactccagtgagatggaaaaaatcccagaaatt |
| | | ggcaaatttggggaaaaagcacctccagctccctctcatgtatggcgtccagcagccttgtttctgactcttc |
| | | tgtgccttctgttgctcattggattgggagtcttggcaagcatgtttcacgtaactttgaagatagaaatgaa |
| | | aaaaatgaacaaactacaaaacatcagtgaagagctccagagaaatatttctctacaactgatgagtaacat |
| | | gaatatctccaacaagatcaggaacctctccaccacactgcaaacaatagccaccaaattatgtcgtgag |
| | | ctatatagcaaagaacaagagcacaaatgtaagccttgtccaaggagatggatttggcataaggacagc |
| | | tgttatttcctaagtgatgatgtccaaacatggcaggagagtaaaatggcctgtgctgctcagaatgccagc |
| | | ctgttgaagataaacaacaaaaatgcattggaatttataaaatcccagagtagatcatatgactattggctg |
| | | ggattatctcctgaagaagattccactcgtggtatgagagtggataatataatcaactcctctgcctgggtta |
| | | taagaaacgcacctgacttaaataacatgtattgtggatatataaatagactatatgttcaatattatcactg |
| | | cacttataaaaaaagaatgatatgtgagaagatggccaatccagtgcagcttggttctacatattttagggag |
| | | gcatga |
| 3 | scFv of Antibody 1 | QVQLVQSGAEVKKPGASVKVSCKASGYTFTSYAMDWVRQAPGQGLEWM |
| | | GAISPSGGGTVYNEKFQGRVTMTRDTSTSTVYMELSSLRSEDTAVYYCAR |
| | | EYWGELFDLWGQGTLVTVSSGGGGSGGGGSGGGGSDIQMTQSPSSLSA |
| | | SVGDRVTITCRASQDIGNWLNWYQQKPGKAPKLLIYGASNLQSGVPSRFS |
| | | GSGSGTDFTLTISSLQPEDFATYYCQQAYGYPFTFGQGTKVEIK |
| 4 | scFv of Antibody 2 | QVQLVQSGAEVKKPGASVKVSCKASGYTFTSYSMSWVRQAPGQGLEWM |
| | | GVISSGTGSTYYNEKFQGRVTMTRDTSTSTVYMELSSLRSEDTAVYYCAKN |
| | | SWGDDFDLWGQGTLVTVSSGGGGGGGGSGGGGSDIQMTQSPSSLSAS |
| | | VGDRVTITCQASQNINNFLAWYQQKPGKAPKLLIYDASNLATGVPSRFSGS |
| | | GSGTDFTLTISSLQPEDFATYYCQQSYNTPLTFGQGTKVEIK |
| 5 | scFv of Antibody 3 | QVQLVQSGAEVKKPGASVKVSCKASGYTFTSYDIDWVRQAPGQGLEWMG |
| | | GISPQSGGTSYNEKFQGRVTMTRDTSTSTVYMELSSLRSEDTAVYYCARV |
| | | HGDYHFDYWGQGTLVTVSSGGGGSGGGGSGGGGSDIQMTQSPSSLSAS |
| | | VGDRVTITCQASQDVSTDLAWYQQKPGKAPKLLIYHASSLESGVPSRFSGS |
| | | GSGTDFTLTISSLQPEDFATYYCQQYYNYPLTFGQGTKVEIK |
| 6 | scFv of Antibody 4 | QVQLVQSGAEVKKPGASVKVSCKASGYTFTSYDMSWVRQAPGQGLEWM |
| | | GAINPSGDFTSYNEKFQGRVTMTRDTSTSTVYMELSSLRSEDTAVYYCAR |
| | | GVGWSFLDYWGQGTLVTVSSGGGGSGGGGSGGGGSDIQMTQSPSSLSA |
| | | SVGDRVTITCQASQDVRTWLAWYQQKPGKAPKLLIYSASSLQTGVPSRFS |
| | | GSGSGTDFTLTISSLQPEDFATYYCQQSNYLPFTFGQGTKVEIK |
| 7 | scFv of Antibody 5 | QVQLVQSGAEVKKPGASVKVSCKASGYTFTDYAMDWVRQAPGQGLEWM |
| | | GWISGQSGSTSYNEKFQGRVTMTRDTSTSTVYMELSSLRSEDTAVYYCAK |
| | | WLDGPWFDVWGQGTLVTVSSGGGGSGGGGSGGGGSDIQMTQSPSSLSA |
| | | SVGDRVTITCKASEDIHSDLAWYQQKPGKAPKLLIYSASSLQSGVPSRFSG |
| | | SGSGTDFTLTISSLQPEDFATYYCQQAYYLPLTFGQGTKVEIK |
| 8 | scFv of Antibody 6 | QVQLVQSGAEVKKPGASVKVSCKASGYTFTDYGMDWVRQAPGQGLEWM |
| | | GYISGGTGRTAYNEKFQGRVTMTRDTSTSTVYMELSSLRSEDTAVYYCAR |
| | | YGANDGFDIWGQGTLVTVSSGGGGSGGGGSGGGGSDIQMTQSPSSLSAS |
| | | VGDRVTITCRASQDISSYLNWYQQKPGKAPKLLIYGASRLYTGVPSRFSGS |
| | | GSGTDFTLTISSLQPEDFATYYCQQGYNTPYTFGGGTKVEIK |
| 9 | scFv of Antibody 7 | QVQLVQSGAEVKKPGASVKVSCKASGYTFTHYAMDWVRQAPGQGLEWM |
| | | GGISPQSGGTSYNEKFQGRVTMTRDTSTSTVYMELSSLRSEDTAVYYCAR |
| | | VHGDYHFDYWGQGTLVTVSSGGGGSGGGGSGGGGSDIQMTQSPSSLSA |
| | | SVGDRVTITCQASQDVSTDLAWYQQKPGKAPKLLIYHASSLESGVPSRFSG |
| | | SGSGTDFTLTISSLQPEDFATYYCQQYYNYPLTFGQGTKVEIK |
| 10 | scFv of Antibody 8 | EVQLLESGGGLVQPGGSLRLSCAASGFTFSSYAMSWVRQAPGKGLEWVS |
| | | AINSQGKSTYYADSVKGRFTISRDNSKNTLYLQMNSLRAEDTAVYYCARPD |
| | | HGDFMDVWGQGTLVTVSSGGGGSGGGGSGGGGSDIQMTQSPSSLSASV |
| | | GDRVTITCRASQGISNWLAWYQQKPGKAPKLLIYGASSLQSGVPSRFSGS |
| | | GSGTDFTLTISSLQPEDFATYYCQQYSSFPTTFGQGTKVEIK |
| 11 | scFv of Antibody 9 | QVQLVQSGAEVKKPGASVKVSCKASGYTFTDYDMSWVRQAPGQGLEWM |
| | | GVISSSGGSTTYNEKFQGRVTMTRDTSTSTVYMELSSLRSEDTAVYYCARS |
| | | PMNEDWFDYWGQGTLVTVSSGGGGSGGGGSGGGGSDIQMTQSPSSLSA |
| | | SVGDRVTITCKASQGVSTWLNWYQQKPGKAPKLLIYVASRLESGVPSRFS |
| | | GSGSGTDFTLTISSLQPEDFATYYCQQSSFLPITFGGGTKVEIK |
| 12 | scFv of Antibody 10 | QVQLVQSGAEVKKPGASVKVSCKASGYTFTHYAMSWVRQAPGQGLEWM |
| | | GGIYGGGDSTNYNEKFQGRVTMTRDTSTSTVYMELSSLRSEDTAVYYCAK |
| | | ASWEDTFDYWGQGTLVTVSSGGGGSGGGGSGGGGSDIQMTQSPSSLSA |
| | | SVGDRVTITCKASQDIRNYLAWYQQKPGKAPKLLIYGASSLASGVPSRFSG |
| | | SGSGTDFTLTISSLQPEDFATYYCQQSYNYPLTFGQGTKVEIK |
| 13 | V_{H} of Antibody 1 | QVQLVQSGAEVKKPGASVKVSCKASGYTFTSYAMDWVRQAPGQGLEWM |
| | | GAISPSGGGTVYNEKFQGRVTMTRDTSTSTVYMELSSLRSEDTAVYYCAR |
| | | EYWGELFDLWGQGTLVTVSS |
| 14 | V_{H} of Antibody 2 | QVQLVQSGAEVKKPGASVKVSCKASGYTFTSYSMSWVRQAPGQGLEWM |
| | | GVISSGTGSTYYNEKFQGRVTMTRDTSTSTVYMELSSLRSEDTAVYYCAKN |
| | | SWGDDFDLWGQGTLVTVSS |
| 15 | V_{H} of Antibody 3 | QVQLVQSGAEVKKPGASVKVSCKASGYTFTSYDIDWVRQAPGQGLEWMG |
| | | GISPQSGGTSYNEKFQGRVTMTRDTSTSTVYMELSSLRSEDTAVYYCARV |
| | | HGDYHFDYWGQGTLVTVSS |
| 16 | V_{H} of Antibody 4 | QVQLVQSGAEVKKPGASVKVSCKASGYTFTSYDMSWVRQAPGQGLEWM |
| | | GAINPSGDFTSYNEKFQGRVTMTRDTSTSTVYMELSSLRSEDTAVYYCAR |
| | | GVGWSFLDYWGQGTLVTVSS |
| 17 | V_{H} of Antibody 5 | QVQLVQSGAEVKKPGASVKVSCKASGYTFTDYAMDWVRQAPGQGLEWM |
| | | GWISGQSGSTSYNEKFQGRVTMTRDTSTSTVYMELSSLRSEDTAVYYCAK |
| | | WLDGPWFDVWGQGTLVTVSS |
| 18 | V_{H} of Antibody 6 | QVQLVQSGAEVKKPGASVKVSCKASGYTFTDYGMDWVRQAPGQGLEWM |
| | | GYISGGTGRTAYNEKFQGRVTMTRDTSTSTVYMELSSLRSEDTAVYYCAR |
| | | YGANDGFDIWGQGTLVTVSS |
| 19 | V_{H} of Antibody 7 | QVQLVQSGAEVKKPGASVKVSCKASGYTFTHYAMDWVRQAPGQGLEWM |
| | | GGISPQSGGTSYNEKFQGRVTMTRDTSTSTVYMELSSLRSEDTAVYYCAR |
| | | VHGDYHFDYWGQGTLVTVSS |
| 20 | V_{H} of Antibody 8 | EVQLLESGGGLVQPGGSLRLSCAASGFTFSSYAMSWVRQAPGKGLEWVS |
| | | AINSQGKSTYYADSVKGRFTISRDNSKNTLYLQMNSLRAEDTAVYYCARPD |
| | | HGDFMDVWGQGTLVTVSS |
| 21 | V_{H} of Antibody 9 | QVQLVQSGAEVKKPGASVKVSCKASGYTFTDYDMSWVRQAPGQGLEWM |
| | | GVISSSGGSTTYNEKFQGRVTMTRDTSTSTVYMELSSLRSEDTAVYYCARS |
| | | PMNEDWFDYWGQGTLVTVSS |
| 22 | V_{H} of Antibody 10 | QVQLVQSGAEVKKPGASVKVSCKASGYTFTHYAMSWVRQAPGQGLEWM |
| | | GGIYGGGDSTNYNEKFQGRVTMTRDTSTSTVYMELSSLRSEDTAVYYCAK |
| | | ASWEDTFDYWGQGTLVTVSS |
| 23 | V_{H} CDR1 of Antibody 1 | SYAMD |
| 24 | V_{H} CDR1 of Antibody 2 | SYSMS |
| 25 | V_{H} CDR1 of Antibody 3 | SYDID |
| 26 | V_{H} CDR1 of Antibody 4 | SYDMS |
| 27 | V_{H} CDR1 of Antibody 5 | DYAMD |
| 28 | V_{H} CDR1 of Antibody 6 | DYGMD |
| 29 | V_{H} CDR1 of Antibody 7 | HYAMD |
| 30 | V_{H} CDR1 of Antibody 8 | SYAMS |
| 31 | V_{H} CDR1 of Antibody 9 | DYDMS |
| 32 | V_{H} CDR1 of Antibody 10 | HYAMS |
| 33 | V_{H} CDR2 of Antibody 1 | AISPSGGGTVYNEKFQG |
| 34 | V_{H} CDR2 of Antibody 2 | VISSGTGSTYYNEKFQG |
| 35 | V_{H} CDR2 of Antibody 3 | GISPQSGGTSYNEKFQG |
| 36 | V_{H} CDR2 of Antibody 4 | AINPSGDFTSYNEKFQG |
| 37 | V_{H} CDR2 of Antibody 5 | WISGQSGSTSYNEKFQG |
| 38 | V_{H} CDR2 of Antibody 6 | YISGGTGRTAYNEKFQG |
| 39 | V_{H} CDR2 of Antibody 7 | GISPQSGGTSYNEKFQG |
| 40 | V_{H} CDR2 of Antibody 8 | AINSQGKSTYYADSVKG |
| 41 | V_{H} CDR2 of Antibody 9 | VISSSGGSTTYNEKFQG |
| 42 | V_{H} CDR2 of Antibody 10 | GIYGGGDSTNYNEKFQG |
| 43 | V_{H} CDR3 of Antibody 1 | REYWGELFDL |
| 44 | V_{H} CDR3 of Antibody 2 | KNSWGDDFDL |
| 45 | V_{H} CDR3 of Antibody 3 | RVHGDYHFDY |
| 46 | V_{H} CDR3 of Antibody 4 | GVGWSFLDY |
| 47 | V_{H} CDR3 of Antibody 5 | KWLDGPWFDV |
| 48 | V_{H} CDR3 of Antibody 6 | RYGANDGFDI |
| 49 | V_{H} CDR3 of Antibody 7 | RVHGDYHFDY |
| 50 | V_{H} CDR3 of Antibody 8 | RPDHGDFMDV |
| 51 | V_{H} CDR3 of Antibody 9 | RSPMNEDWFDY |
| 52 | V_{H} CDR3 of Antibody 10 | KASWEDTFDY |
| 53 | V_{L} of Antibody 1 | DIQMTQSPSSLSASVGDRVTITCRASQDIGNWLNWYQQKPGKAPKLLIYGA |
| | | SNLQSGVPSRFSGSGSGTDFTLTISSLQPEDFATYYCQQAYGYPFTFGQGT |
| | | KVEIK |
| 54 | V_{L} of Antibody 2 | DIQMTQSPSSLSASVGDRVTITCQASQNINNFLAWYQQKPGKAPKLLIYDAS |
| | | NLATGVPSRFSGSGSGTDFTLTISSLQPEDFATYYCQQSYNTPLTFGQGTK |
| | | VEIK |
| 55 | V_{L} of Antibody 3 | DIQMTQSPSSLSASVGDRVTITCQASQDVSTDLAWYQQKPGKAPKLLIYHA |
| | | SSLESGVPSRFSGSGSGTDFTLTISSLQPEDFATYYCQQYYNYPLTFGQGT |
| | | KVEIK |
| 56 | V_{L} of Antibody 4 | DIQMTQSPSSLSASVGDRVTITCQASQDVRTWLAWYQQKPGKAPKLLIYSA |
| | | SSLQTGVPSRFSGSGSGTDFTLTISSLQPEDFATYYCQQSNYLPFTFGQGT |
| | | KVEIK |
| 57 | V_{L} of Antibody 5 | DIQMTQSPSSLSASVGDRVTITCKASEDIHSDLAWYQQKPGKAPKLLIYSAS |
| | | SLQSGVPSRFSGSGSGTDFTLTISSLQPEDFATYYCQQAYYLPLTFGQGTK |
| | | VEIK |
| 58 | V_{L} of Antibody 6 | DIQMTQSPSSLSASVGDRVTITCRASQDISSYLNWYQQKPGKAPKLLIYGAS |
| | | RLYTGVPSRFSGSGSGTDFTLTISSLQPEDFATYYCQQGYNTPYTFGGGTK |
| | | VEIK |
| 59 | V_{L} of Antibody 7 | DIQMTQSPSSLSASVGDRVTITCQASQDVSTDLAWYQQKPGKAPKLLIYHA |
| | | SSLESGVPSRFSGSGSGTDFTLTISSLQPEDFATYYCQQYYNYPLTFGQGT |
| | | KVEIK |
| 60 | V_{L} of Antibody 8 | DIQMTQSPSSLSASVGDRVTITCRASQGISNWLAWYQQKPGKAPKLLIYGA |
| | | SSLQSGVPSRFSGSGSGTDFTLTISSLQPEDFATYYCQQYSSFPTTFGQGT |
| | | KVEIK |
| 61 | V_{L} of Antibody 9 | DIQMTQSPSSLSASVGDRVTITCKASQGVSTWLNWYQQKPGKAPKLLIYVA |
| | | SRLESGVPSRFSGSGSGTDFTLTISSLQPEDFATYYCQQSSFLPITFGGGTK |
| | | VEIK |
| 62 | VL of Antibody 10 | DIQMTQSPSSLSASVGDRVTITCKASQDIRNYLAWYQQKPGKAPKLLIYGAS |
| | | SLASGVPSRFSGSGSGTDFTLTISSLQPEDFATYYCQQSYNYPLTFGQGTK |
| | | VEIK |
| 63 | V_{L} CDR1 of Antibody 1 | RASQDIGNWLN |
| 64 | V_{L} CDR1 of Antibody 2 | QASQNINNFLA |
| 65 | V_{L} CDR1 of Antibody 3 | QASQDVSTDLA |
| 66 | V_{L} CDR1 of Antibody 4 | QASQDVRTWLA |
| 67 | V_{L} CDR1 of Antibody 5 | KASEDIHSDLA |
| 68 | V_{L} CDR1 of Antibody 6 | RASQDISSYLN |
| 69 | V_{L} CDR1 of Antibody 7 | QASQDVSTDLA |
| 70 | V_{L} CDR1 of Antibody 8 | RASQGISNWLA |
| 71 | V_{L} CDR1 of Antibody 9 | KASQGVSTWLN |
| 72 | V_{L} CDR1 of Antibody 10 | KASQDIRNYLA |
| 73 | V_{L} CDR2 of Antibody 1 | GASNLQS |
| 74 | V_{L} CDR2 of Antibody 2 | DASNLAT |
| 75 | V_{L} CDR2 of Antibody 3 | HASSLES |
| 76 | V_{L} CDR2 of Antibody 4 | SASSLQT |
| 77 | V_{L} CDR2 of Antibody 5 | SASSLQS |
| 78 | V_{L} CDR2 of Antibody 6 | GASRLYT |
| 79 | V_{L} CDR2 of Antibody 7 | HASSLES |
| 80 | V_{L} CDR2 of Antibody 8 | GASSLQS |
| 81 | V_{L} CDR2 of Antibody 9 | VASRLES |
| 82 | V_{L} CDR2 of Antibody 10 | GASSLAS |
| 83 | V_{L} CDR3 of Antibody 1 | QQAYGYPFT |
| 84 | V_{L} CDR3 of Antibody 2 | QQSYNTPLT |
| 85 | V_{L} CDR3 of Antibody 3 | QQYYNYPLT |
| 86 | V_{L} CDR3 of Antibody 4 | QQSNYLPFT |
| 87 | V_{L} CDR3 of Antibody 5 | QQAYYLPLT |
| 88 | V_{L} CDR3 of Antibody 6 | QQGYNTPYT |
| 89 | V_{L} CDR3 of Antibody 7 | QQYYNYPLT |
| 90 | V_{L} CDR3 of Antibody 8 | QQYSSFPTT |
| 91 | V_{L} CDR3 of Antibody 9 | QQSSFLPIT |
| 92 | V_{L} CDR3 of Antibody 10 | QQSYNYPLT |
| 93 | CD8A-CAR comprising | MALPVTALLLPLALLLHAARPQVQLVQSGAEVKKPGASVKVSCKASGYTFT |
| | Antibody 1 | SYAMDWVRQAPGQGLEWMGAISPSGGGTVYNEKFQGRVTMTRDTSTSTV |
| | | YMELSSLRSEDTAVYYCAREYWGELFDLWGQGTLVTVSSGGGGSGGGGS |
| | | GGGGSDIQMTQSPSSLSASVGDRVTITCRASQDIGNWLNWYQQKPGKAPK |
| | | LLIYGASNLQSGVPSRFSGSGSGTDFTLTISSLQPEDFATYYCQQAYGYPFT |
| | | FGQGTKVEIKTTTPAPRPPTPAPTIASQPLSLRPEACRPAAGGAVHTRGLDF |
| | | ACDIYIWAPLAGTCGVLLLSLVITLYCNHRNKRGRKKLLYIFKQPFMRPVQTT |
| | | QEEDGCSCRFPEEEEGGCELRVKFSRSADAPAYQQGQNQLYNELNLGRR |
| | | EEYDVLDKRRGRDPEMGGKPQRRKNPQEGLYNELQKDKMAEAYSEIGMK |
| | | GERRRGKGHDGLYQGLSTATKDTYDALHMQALPPR |
| 94 | CD8A-CAR comprising | MALPVTALLLPLALLLHAARPQVQLVQSGAEVKKPGASVKVSCKASGYTFT |
| | Antibody 2 | SYSMSWVRQAPGQGLEWMGVISSGTGSTYYNEKFQGRVTMTRDTSTSTV |
| | | YMELSSLRSEDTAVYYCAKNSWGDDFDLWGQGTLVTVSSGGGGGGGG |
| | | SGGGGSDIQMTQSPSSLSASVGDRVTITCQASQNINNFLAWYQQKPGKAP |
| | | KLLIYDASNLATGVPSRFSGSGSGTDFTLTISSLQPEDFATYYCQQSYNTPL |
| | | TFGQGTKVEIKTTTPAPRPPTPAPTIASQPLSLRPEACRPAAGGAVHTRGLD |
| | | FACDIYIWAPLAGTCGVLLLSLVITLYCNHRNKRGRKKLLYIFKQPFMRPVQT |
| | | TQEEDGCSCRFPEEEEGGCELRVKFSRSADAPAYQQGQNQLYNELNLGR |
| | | REEYDVLDKRRGRDPEMGGKPQRRKNPQEGLYNELQKDKMAEAYSEIGM |
| | | KGERRRGKGHDGLYQGLSTATKDTYDALHMQALPPR |
| 95 | CD8A-CAR comprising | MALPVTALLLPLALLLHAARPQVQLVQSGAEVKKPGASVKVSCKASGYTFT |
| | Antibody 3 | SYDIDWVRQAPGQGLEWMGGISPQSGGTSYNEKFQGRVTMTRDTSTSTV |
| | | YMELSSLRSEDTAVYYCARVHGDYHFDYWGQGTLVTVSSGGGGSGGGG |
| | | SGGGGSDIQMTQSPSSLSASVGDRVTITCQASQDVSTDLAWYQQKPGKAP |
| | | KLLIYHASSLESGVPSRFSGSGSGTDFTLTISSLQPEDFATYYCQQYYNYPL |
| | | TFGQGTKVEIKTTTPAPRPPTPAPTIASQPLSLRPEACRPAAGGAVHTRGLD |
| | | FACDIYIWAPLAGTCGVLLLSLVITLYCNHRNKRGRKKLLYIFKQPFMRPVQT |
| | | TQEEDGCSCRFPEEEEGGCELRVKFSRSADAPAYQQGQNQLYNELNLGR |
| | | REEYDVLDKRRGRDPEMGGKPQRRKNPQEGLYNELQKDKMAEAYSEIGM |
| | | KGERRRGKGHDGLYQGLSTATKDTYDALHMQALPPR |
| 96 | CD8A-CAR comprising | MALPVTALLLPLALLLHAARPQVQLVQSGAEVKKPGASVKVSCKASGYTFT |
| | Antibody 4 | SYDMSWVRQAPGQGLEWMGAINPSGDFTSYNEKFQGRVTMTRDTSTSTV |
| | | YMELSSLRSEDTAVYYCARGVGWSFLDYWGQGTLVTVSSGGGGSGGGG |
| | | SGGGGSDIQMTQSPSSLSASVGDRVTITCQASQDVRTWLAWYQQKPGKA |
| | | PKLLIYSASSLQTGVPSRFSGSGSGTDFTLTISSLQPEDFATYYCQQSNYLP |
| | | FTFGQGTKVEIKTTTPAPRPPTPAPTIASQPLSLRPEACRPAAGGAVHTRGL |
| | | DFACDIYIWAPLAGTCGVLLLSLVITLYCNHRNKRGRKKLLYIFKQPFMRPVQ |
| | | TTQEEDGCSCRFPEEEEGGCELRVKFSRSADAPAYQQGQNQLYNELNLG |
| | | RREEYDVLDKRRGRDPEMGGKPQRRKNPQEGLYNELQKDKMAEAYSEIG |
| | | MKGERRRGKGHDGLYQGLSTATKDTYDALHMQALPPR |
| 97 | CD8A-CAR comprising | MALPVTALLLPLALLLHAARPQVQLVQSGAEVKKPGASVKVSCKASGYTFT |
| | Antibody 5 | DYAMDWVRQAPGQGLEWMGWISGQSGSTSYNEKFQGRVTMTRDTSTST |
| | | VYMELSSLRSEDTAVYYCAKWLDGPWFDVWGQGTLVTVSSGGGGSGGG |
| | | GSGGGGSDIQMTQSPSSLSASVGDRVTITCKASEDIHSDLAWYQQKPGKA |
| | | PKLLIYSASSLQSGVPSRFSGSGSGTDFTLTISSLQPEDFATYYCQQAYYLP |
| | | LTFGQGTKVEIKTTTPAPRPPTPAPTIASQPLSLRPEACRPAAGGAVHTRGL |
| | | DFACDIYIWAPLAGTCGVLLLSLVITLYCNHRNKRGRKKLLYIFKQPFMRPVQ |
| | | TTQEEDGCSCRFPEEEEGGCELRVKFSRSADAPAYQQGQNQLYNELNLG |
| | | RREEYDVLDKRRGRDPEMGGKPQRRKNPQEGLYNELQKDKMAEAYSEIG |
| | | MKGERRRGKGHDGLYQGLSTATKDTYDALHMQALPPR |
| 98 | CD8A-CAR comprising | MALPVTALLLPLALLLHAARPQVQLVQSGAEVKKPGASVKVSCKASGYTFT |
| | Antibody 6 | DYGMDWVRQAPGQGLEWMGYISGGTGRTAYNEKFQGRVTMTRDTSTST |
| | | VYMELSSLRSEDTAVYYCARYGANDGFDIWGQGTLVTVSSGGGGSGGGG |
| | | SGGGGSDIQMTQSPSSLSASVGDRVTITCRASQDISSYLNWYQQKPGKAP |
| | | KLLIYGASRLYTGVPSRFSGSGSGTDFTLTISSLQPEDFATYYCQQGYNTPY |
| | | TFGGGTKVEIKTTTPAPRPPTPAPTIASQPLSLRPEACRPAAGGAVHTRGLD |
| | | FACDIYIWAPLAGTCGVLLLSLVITLYCNHRNKRGRKKLLYIFKQPFMRPVQT |
| | | TQEEDGCSCRFPEEEEGGCELRVKFSRSADAPAYQQGQNQLYNELNLGR |
| | | REEYDVLDKRRGRDPEMGGKPQRRKNPQEGLYNELQKDKMAEAYSEIGM |
| | | KGERRRGKGHDGLYQGLSTATKDTYDALHMQALPPR |
| 99 | CD8A-CAR comprising | MALPVTALLLPLALLLHAARPQVQLVQSGAEVKKPGASVKVSCKASGYTFT |
| | Antibody 7 | HYAMDWVRQAPGQGLEWMGGISPQSGGTSYNEKFQGRVTMTRDTSTST |
| | | VYMELSSLRSEDTAVYYCARVHGDYHFDYWGQGTLVTVSSGGGGSGGG |
| | | GSGGGGSDIQMTQSPSSLSASVGDRVTITCQASQDVSTDLAWYQQKPGK |
| | | APKLLIYHASSLESGVPSRFSGSGSGTDFTLTISSLQPEDFATYYCQQYYNY |
| | | PLTFGQGTKVEIKTTTPAPRPPTPAPTIASQPLSLRPEACRPAAGGAVHTRG |
| | | LDFACDIYIWAPLAGTCGVLLLSLVITLYCNHRNKRGRKKLLYIFKQPFMRPV |
| | | QTTQEEDGCSCRFPEEEEGGCELRVKFSRSADAPAYQQGQNQLYNELNL |
| | | GRREEYDVLDKRRGRDPEMGGKPQRRKNPQEGLYNELQKDKMAEAYSEI |
| | | GMKGERRRGKGHDGLYQGLSTATKDTYDALHMQALPPR |
| 100 | CD8A-CAR comprising | MALPVTALLLPLALLLHAARPEVQLLESGGGLVQPGGSLRLSCAASGFTFSS |
| | Antibody 8 | YAMSWVRQAPGKGLEWVSAINSQGKSTYYADSVKGRFTISRDNSKNTLYL |
| | | QMNSLRAEDTAVYYCARPDHGDFMDVWGQGTLVTVSSGGGGSGGGGSG |
| | | GGGSDIQMTQSPSSLSASVGDRVTITCRASQGISNWLAWYQQKPGKAPKL |
| | | LIYGASSLQSGVPSRFSGSGSGTDFTLTISSLQPEDFATYYCQQYSSFPTTF |
| | | GQGTKVEIKTTTPAPRPPTPAPTIASQPLSLRPEACRPAAGGAVHTRGLDFA |
| | | CDIYIWAPLAGTCGVLLLSLVITLYCNHRNKRGRKKLLYIFKQPFMRPVQTTQ |
| | | EEDGCSCRFPEEEEGGCELRVKFSRSADAPAYQQGQNQLYNELNLGRRE |
| | | EYDVLDKRRGRDPEMGGKPQRRKNPQEGLYNELQKDKMAEAYSEIGMKG |
| | | ERRRGKGHDGLYQGLSTATKDTYDALHMQALPPR |
| 101 | CD8A-CAR comprising | MALPVTALLLPLALLLHAARPQVQLVQSGAEVKKPGASVKVSCKASGYTFT |
| | Antibody 9 | DYDMSWVRQAPGQGLEWMGVISSSGGSTTYNEKFQGRVTMTRDTSTSTV |
| | | YMELSSLRSEDTAVYYCARSPMNEDWFDYWGQGTLVTVSSGGGGSGGG |
| | | GSGGGGSDIQMTQSPSSLSASVGDRVTITCKASQGVSTWLNWYQQKPGK |
| | | APKLLIYVASRLESGVPSRFSGSGSGTDFTLTISSLQPEDFATYYCQQSSFL |
| | | PITFGGGTKVEIKTTTPAPRPPTPAPTIASQPLSLRPEACRPAAGGAVHTRG |
| | | LDFACDIYIWAPLAGTCGVLLLSLVITLYCNHRNKRGRKKLLYIFKQPFMRPV |
| | | QTTQEEDGCSCRFPEEEEGGCELRVKFSRSADAPAYQQGQNQLYNELNL |
| | | GRREEYDVLDKRRGRDPEMGGKPQRRKNPQEGLYNELQKDKMAEAYSEI |
| | | GMKGERRRGKGHDGLYQGLSTATKDTYDALHMQALPPR |
| 102 | CD8A-CAR comprising | MALPVTALLLPLALLLHAARPQVQLVQSGAEVKKPGASVKVSCKASGYTFT |
| | Antibody 10 | HYAMSWVRQAPGQGLEWMGGIYGGGDSTNYNEKFQGRVTMTRDTSTST |
| | | VYMELSSLRSEDTAVYYCAKASWEDTFDYWGQGTLVTVSSGGGGSGGGG |
| | | SGGGGSDIQMTQSPSSLSASVGDRVTITCKASQDIRNYLAWYQQKPGKAP |
| | | KLLIYGASSLASGVPSRFSGSGSGTDFTLTISSLQPEDFATYYCQQSYNYPL |
| | | TFGQGTKVEIKTTTPAPRPPTPAPTIASQPLSLRPEACRPAAGGAVHTRGLD |
| | | FACDIYIWAPLAGTCGVLLLSLVITLYCNHRNKRGRKKLLYIFKQPFMRPVQT |
| | | TQEEDGCSCRFPEEEEGGCELRVKFSRSADAPAYQQGQNQLYNELNLGR |
| | | REEYDVLDKRRGRDPEMGGKPQRRKNPQEGLYNELQKDKMAEAYSEIGM |
| | | KGERRRGKGHDGLYQGLSTATKDTYDALHMQALPPR |
| 103 | CD28-CAR comprising | MALPVTALLLPLALLLHAARPQVQLVQSGAEVKKPGASVKVSCKASGYTFT |
| | Antibody 1 | SYAMDWVRQAPGQGLEWMGAISPSGGGTVYNEKFQGRVTMTRDTSTSTV |
| | | YMELSSLRSEDTAVYYCAREYWGELFDLWGQGTLVTVSSGGGGSGGGGS |
| | | GGGGSDIQMTQSPSSLSASVGDRVTITCRASQDIGNWLNWYQQKPGKAPK |
| | | LLIYGASNLQSGVPSRFSGSGSGTDFTLTISSLQPEDFATYYCQQAYGYPFT |
| | | FGQGTKVEIKLDNEKSNGTIIHVKGKHLCPSPLFPGPSKPFWVLVVVGGVLA |
| | | CYSLLVTVAFIIFWVRSKRSRLLHSDYMNMTPRRPGPTRKHYQPYAPPRDF |
| | | AAYRSRVKFSRSADAPAYQQGQNQLYNELNLGRREEYDVLDKRRGRDPE |
| | | MGGKPQRRKNPQEGLYNELQKDKMAEAYSEIGMKGERRRGKGHDGLYQ |
| | | GLSTATKDTYDALHMQALPPR |
| 104 | CD28-CAR comprising | MALPVTALLLPLALLLHAARPQVQLVQSGAEVKKPGASVKVSCKASGYTFT |
| | Antibody 2 | SYSMSWVRQAPGQGLEWMGVISSGTGSTYYNEKFQGRVTMTRDTSTSTV |
| | | YMELSSLRSEDTAVYYCAKNSWGDDFDLWGQGTLVTVSSGGGGSGGGG |
| | | SGGGGSDIQMTQSPSSLSASVGDRVTITCQASQNINNFLAWYQQKPGKAP |
| | | KLLIYDASNLATGVPSRFSGSGSGTDFTLTISSLQPEDFATYYCQQSYNTPL |
| | | TFGQGTKVEIKLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPQRRKNPQEGLYNELQKDKMAEAYSEIGMKGERRRGKGHDGLY |
| | | QGLSTATKDTYDALHMQALPPR |
| 105 | CD28-CAR comprising | MALPVTALLLPLALLLHAARPQVQLVQSGAEVKKPGASVKVSCKASGYTFT |
| | Antibody 3 | SYDIDWVRQAPGQGLEWMGGISPQSGGTSYNEKFQGRVTMTRDTSTSTV |
| | | YMELSSLRSEDTAVYYCARVHGDYHFDYWGQGTLVTVSSGGGGSGGGG |
| | | SGGGGSDIQMTQSPSSLSASVGDRVTITCQASQDVSTDLAWYQQKPGKAP |
| | | KLLIYHASSLESGVPSRFSGSGSGTDFTLTISSLQPEDFATYYCQQYYNYPL |
| | | TFGQGTKVEIKLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPQRRKNPQEGLYNELQKDKMAEAYSEIGMKGERRRGKGHDGLY |
| | | QGLSTATKDTYDALHMQALPPR |
| 106 | CD28-CAR comprising | MALPVTALLLPLALLLHAARPQVQLVQSGAEVKKPGASVKVSCKASGYTFT |
| | Antibody 4 | SYDMSWVRQAPGQGLEWMGAINPSGDFTSYNEKFQGRVTMTRDTSTSTV |
| | | YMELSSLRSEDTAVYYCARGVGWSFLDYWGQGTLVTVSSGGGGSGGGG |
| | | SGGGGSDIQMTQSPSSLSASVGDRVTITCQASQDVRTWLAWYQQKPGKA |
| | | PKLLIYSASSLQTGVPSRFSGSGSGTDFTLTISSLQPEDFATYYCQQSNYLP |
| | | FTFGQGTKVEIKLDNEKSNGTIIHVKGKHLCPSPLFPGPSKPFWVLVVVGGV |
| | | LACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRD |
| | | PEMGGKPQRRKNPQEGLYNELQKDKMAEAYSEIGMKGERRRGKGHDGLY |
| | | QGLSTATKDTYDALHMQALPPR |
| 107 | CD28-CAR comprising | MALPVTALLLPLALLLHAARPQVQLVQSGAEVKKPGASVKVSCKASGYTFT |
| | Antibody 5 | DYAMDWVRQAPGQGLEWMGWISGQSGSTSYNEKFQGRVTMTRDTSTST |
| | | VYMELSSLRSEDTAVYYCAKWLDGPWFDVWGQGTLVTVSSGGGGSGGG |
| | | GSGGGGSDIQMTQSPSSLSASVGDRVTITCKASEDIHSDLAWYQQKPGKA |
| | | PKLLIYSASSLQSGVPSRFSGSGSGTDFTLTISSLQPEDFATYYCQQAYYLP |
| | | LTFGQGTKVEIKLDNEKSNGTIIHVKGKHLCPSPLFPGPSKPFWVLVVVGGV |
| | | LACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRD |
| | | PEMGGKPQRRKNPQEGLYNELQKDKMAEAYSEIGMKGERRRGKGHDGLY |
| | | QGLSTATKDTYDALHMQALPPR |
| 108 | CD28-CAR comprising | MALPVTALLLPLALLLHAARPQVQLVQSGAEVKKPGASVKVSCKASGYTFT |
| | Antibody 6 | DYGMDWVRQAPGQGLEWMGYISGGTGRTAYNEKFQGRVTMTRDTSTST |
| | | VYMELSSLRSEDTAVYYCARYGANDGFDIWGQGTLVTVSSGGGGSGGGG |
| | | SGGGGSDIQMTQSPSSLSASVGDRVTITCRASQDISSYLNWYQQKPGKAP |
| | | KLLIYGASRLYTGVPSRFSGSGSGTDFTLTISSLQPEDFATYYCQQGYNTPY |
| | | TFGGGTKVEIKLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPQRRKNPQEGLYNELQKDKMAEAYSEIGMKGERRRGKGHDGLY |
| | | QGLSTATKDTYDALHMQALPPR |
| 109 | CD28-CAR comprising | MALPVTALLLPLALLLHAARPQVQLVQSGAEVKKPGASVKVSCKASGYTFT |
| | Antibody 7 | HYAMDWVRQAPGQGLEWMGGISPQSGGTSYNEKFQGRVTMTRDTSTST |
| | | VYMELSSLRSEDTAVYYCARVHGDYHFDYWGQGTLVTVSSGGGGSGGG |
| | | GSGGGGSDIQMTQSPSSLSASVGDRVTITCQASQDVSTDLAWYQQKPGK |
| | | APKLLIYHASSLESGVPSRFSGSGSGTDFTLTISSLQPEDFATYYCQQYYNY |
| | | PLTFGQGTKVEIKLDNEKSNGTIIHVKGKHLCPSPLFPGPSKPFWVLVVVGG |
| | | VLACYSLLVTVAFIIFWVRSKRSRLLHSDYMNMTPRRPGPTRKHYQPYAPP |
| | | RDFAAYRSRVKFSRSADAPAYQQGQNQLYNELNLGRREEYDVLDKRRGR |
| | | DPEMGGKPQRRKNPQEGLYNELQKDKMAEAYSEIGMKGERRRGKGHDGL |
| | | YQGLSTATKDTYDALHMQALPPR |
| 110 | CD28-CAR comprising | MALPVTALLLPLALLLHAARPEVQLLESGGGLVQPGGSLRLSCAASGFTFSS |
| | Antibody 8 | YAMSWVRQAPGKGLEWVSAINSQGKSTYYADSVKGRFTISRDNSKNTLYL |
| | | QMNSLRAEDTAVYYCARPDHGDFMDVWGQGTLVTVSSGGGGSGGGGSG |
| | | GGGSDIQMTQSPSSLSASVGDRVTITCRASQGISNWLAWYQQKPGKAPKL |
| | | LIYGASSLQSGVPSRFSGSGSGTDFTLTISSLQPEDFATYYCQQYSSFPTTF |
| | | GQGTKVEIKLDNEKSNGTIIHVKGKHLCPSPLFPGPSKPFWVLVVVGGVLAC |
| | | YSLLVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDPEM |
| | | GGKPQRRKNPQEGLYNELQKDKMAEAYSEIGMKGERRRGKGHDGLYQGL |
| | | STATKDTYDALHMQALPPR |
| 111 | CD28-CAR comprising | MALPVTALLLPLALLLHAARPQVQLVQSGAEVKKPGASVKVSCKASGYTFT |
| | Antibody 9 | DYDMSWVRQAPGQGLEWMGVISSSGGSTTYNEKFQGRVTMTRDTSTSTV |
| | | YMELSSLRSEDTAVYYCARSPMNEDWFDYWGQGTLVTVSSGGGGSGGG |
| | | GSGGGGSDIQMTQSPSSLSASVGDRVTITCKASQGVSTWLNWYQQKPGK |
| | | APKLLIYVASRLESGVPSRFSGSGSGTDFTLTISSLQPEDFATYYCQQSSFL |
| | | PITFGGGTKVEIKLDNEKSNGTIIHVKGKHLCPSPLFPGPSKPFWVLVVVGG |
| | | VLACYSLLVTVAFIIFWVRSKRSRLLHSDYMNMTPRRPGPTRKHYQPYAPP |
| | | RDFAAYRSRVKFSRSADAPAYQQGQNQLYNELNLGRREEYDVLDKRRGR |
| | | DPEMGGKPQRRKNPQEGLYNELQKDKMAEAYSEIGMKGERRRGKGHDGL |
| | | YQGLSTATKDTYDALHMQALPPR |
| 112 | CD28-CAR comprising | MALPVTALLLPLALLLHAARPQVQLVQSGAEVKKPGASVKVSCKASGYTFT |
| | Antibody 10 | HYAMSWVRQAPGQGLEWMGGIYGGGDSTNYNEKFQGRVTMTRDTSTST |
| | | VYMELSSLRSEDTAVYYCAKASWEDTFDYWGQGTLVTVSSGGGGSGGGG |
| | | SGGGGSDIQMTQSPSSLSASVGDRVTITCKASQDIRNYLAWYQQKPGKAP |
| | | KLLIYGASSLASGVPSRFSGSGSGTDFTLTISSLQPEDFATYYCQQSYNYPL |
| | | TFGQGTKVEIKLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPQRRKNPQEGLYNELQKDKMAEAYSEIGMKGERRRGKGHDGLY |
| | | QGLSTATKDTYDALHMQALPPR |
| 113 | Human CD247 (CD3-zeta) | RVKFSRSADAPAYQQGQNQLYNELNLGRREEYDVLDKRRGRDPEMGGKP |
| | cytoplasmic domain | QRRKNPQEGLYNELQKDKMAEAYSEIGMKGERRRGKGHDGLYQGLSTAT |
| | | KDTYDALHMQALPPR |
| 114 | Human | KRGRKKLLYIFKQPFMRPVQTTQEEDGCSCRFPEEEEGGCEL |
| | CD137 co-stimulatory | |
| | domain | |
| 115 | Human CD8A | IYIWAPLAGTCGVLLLSLVITLYCNHRN |
| | transmembrane domai | |
| 116 | Human CD8A hinge | TTTPAPRPPTPAPTIASQPLSLRPEACRPAAGGAVHTRGLDFACD |
| | domain | |
| 117 | Linker 1 | GGGGGGGGSGGGGS |
| 118 | Linker 2 | GGGGSGGGGSGGGGSGGGGS |
| 119 | CD8A extracellular | MALPVTALLLPLALLLHAARP |
| | signal peptide | |
| 120 | Human CD28 co- | RSKRSRLLHSDYMNMTPRRPGPTRKHYQPYAPPRDFAAYRS |
| | stimulatory domain | |
| 121 | Human CD28 | FWVLVVVGGVLACYSLLVTVAFIIFWV |
| | transmembrane domain | |
| 122 | Human CD28 hinge | LDNEKSNGTIIHVKGKHLCPSPLFPGPSKP |
| | domain | |
| 123 | V_{H} CDR1 of antibody | SGYTFTRYTMH |
| | fragment that binds | |
| | CD3 | |
| 124 | V_{H} CDR2 of antibody | YINPSRGYTNYNQKFKD |
| | fragment | |
| | that binds CD3 | |
| 125 | V_{H} CDR3 of antibody | YYDDHYCL |
| | fragment | |
| | that binds CD3 | |
| 126 | V_{L} CDR1 of antibody | SASSSVSYMN |
| | fragment | |
| | that binds CD3 | |
| 127 | V_{L} CDR2 of antibody | DTSKLAS |
| | fragment | |
| | that binds CD3 | |
| 128 | V_{L} CDR3 of antibody | QQWSSNPF |
| | fragment | |
| | that binds CD3 | |
| 129 | CD8A-CAR sequence of | MALPVTALLLPLALLLHAARPQVQLQESGPGLVKPSETLSLTCTVSGGSISS |
| | Comparative CAR 11 | YYWSWIRQPPGKGLEWIGYIYYSGSTNYNPSLKSRVTISVDTSKNQFSLKLS |
| | | SVTAADTAVYYCVSLVYCGGDCYSGFDYWGQGTLVTVSSGGGGSGGGG |
| | | SGGGGSDIQLTQSPSSLSASVGDRVSFTCQASQDINNFLNWYQQKPGKAP |
| | | KLLIYDASNLETGVPSRFSGSGSGTDFTFTISSLQPEDIATYYCQQYGNLPF |
| | | TFGGGTKVEIKRTTTPAPRPPTPAPTIASQPLSLRPEACRPAAGGAVHTRGL |
| | | DFACDIYIWAPLAGTCGVLLLSLVITLYCNHRNKRGRKKLLYIFKQPFMRPVQ |
| | | TTQEEDGCSCRFPEEEEGGCELRVKFSRSADAPAYQQGQNQLYNELNLG |
| | | RREEYDVLDKRRGRDPEMGGKPQRRKNPQEGLYNELQKDKMAEAYSEIG |
| | | MKGERRRGKGHDGLYQGLSTATKDTYDALHMQALPPR |
| 130 | CD28-CAR sequence of | MALPVTALLLPLALLLHAARPQVQLQESGPGLVKPSETLSLTCTVSGGSISS |
| | Comparative CAR 11 | YYWSWIRQPPGKGLEWIGYIYYSGSTNYNPSLKSRVTISVDTSKNQFSLKLS |
| | | SVTAADTAVYYCVSLVYCGGDCYSGFDYWGQGTLVTVSSGGGGSGGGG |
| | | SGGGGSDIQLTQSPSSLSASVGDRVSFTCQASQDINNFLNWYQQKPGKAP |
| | | KLLIYDASNLETGVPSRFSGSGSGTDFTFTISSLQPEDIATYYCQQYGNLPF |
| | | TFGGGTKVEIKRLDNEKSNGTIIHVKGKHLCPSPLFPGPSKPFWVLVVVGGV |
| | | LACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRD |
| | | PEMGGKPQRRKNPQEGLYNELQKDKMAEAYSEIGMKGERRRGKGHDGLY |
| | | QGLSTATKDTYDALHMQALPPR |
| 131 | CD8A-CAR sequence of | MALPVTALLLPLALLLHAARPEVQLQQSGPELVKPGASVKMSCKASGYTFT |
| | Comparative CAR 12 | SYFIHWVKQKPGQGLEWIGFINPYNDGSKYNEKFKGKATLTSDKSSSTAYM |
| | | ELSSLTSEDSAVYYCTRDDGYYGYAMDYWGQGTSVTVSSGGGGSGGGG |
| | | SGGGGSDIQMTQSPSSLSASLGERVSLTCRATQELSGYLSWLQQKPDGTI |
| | | KRLIYAASTLDSGVPKRFSGNRSGSDYSLTISSLESEDFADYYCLQYAIYPYT |
| | | FGGGTKLEIKRTTTPAPRPPTPAPTIASQPLSLRPEACRPAAGGAVHTRGLD |
| | | FACDIYIWAPLAGTCGVLLLSLVITLYCNHRNKRGRKKLLYIFKQPFMRPVQT |
| | | TQEEDGCSCRFPEEEEGGCELRVKFSRSADAPAYQQGQNQLYNELNLGR |
| | | REEYDVLDKRRGRDPEMGGKPQRRKNPQEGLYNELQKDKMAEAYSEIGM |
| | | KGERRRGKGHDGLYQGLSTATKDTYDALHMQALPPR |
| 132 | CD28-CAR sequence of | MALPVTALLLPLALLLHAARPEVQLQQSGPELVKPGASVKMSCKASGYTFT |
| | Comparative CAR 12 | SYFIHWVKQKPGQGLEWIGFINPYNDGSKYNEKFKGKATLTSDKSSSTAYM |
| | | ELSSLTSEDSAVYYCTRDDGYYGYAMDYWGQGTSVTVSSGGGGSGGGG |
| | | SGGGGSDIQMTQSPSSLSASLGERVSLTCRATQELSGYLSWLQQKPDGTI |
| | | KRLIYAASTLDSGVPKRFSGNRSGSDYSLTISSLESEDFADYYCLQYAIYPYT |
| | | FGGGTKLEIKRLDNEKSNGTIIHVKGKHLCPSPLFPGPSKPFWVLVVVGGVL |
| | | ACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDP |
| | | EMGGKPQRRKNPQEGLYNELQKDKMAEAYSEIGMKGERRRGKGHDGLY |
| | | QGLSTATKDTYDALHMQALPPR |
| 133 | scFv of Comparative | QVQLQESGPGLVKPSETLSLTCTVSGGSISSYYWSWIRQPPGKGLEWIGYI |
| | Antibody 11 | YYSGSTNYNPSLKSRVTISVDTSKNQFSLKLSSVTAADTAVYYCVSLVYCG |
| | | GDCYSGFDYWGQGTLVTVSSGGGGSGGGGGGGGSDIQLTQSPSSLSA |
| | | SVGDRVSFTCQASQDINNFLNWYQQKPGKAPKLLIYDASNLETGVPSRFSG |
| | | SGSGTDFTFTISSLQPEDIATYYCQQYGNLPFTFGGGTKVEIKR |
| 134 | scFv of Comparative | EVQLQQSGPELVKPGASVKMSCKASGYTFTSYFIHWVKQKPGQGLEWIGFI |
| | Antibody 12 | NPYNDGSKYNEKFKGKATLTSDKSSSTAYMELSSLTSEDSAVYYCTRDDG |
| | | YYGYAMDYWGQGTSVTVSSGGGGSGGGGSGGGGSDIQMTQSPSSLSAS |
| | | LGERVSLTCRATQELSGYLSWLQQKPDGTIKRLIYAASTLDSGVPKRFSGN |
| | | RSGSDYSLTISSLESEDFADYYCLQYAIYPYTFGGGTKLEIKR |
| 135 | V_{H} of Comparative | QVQLQESGPGLVKPSETLSLTCTVSGGSISSYYWSWIRQPPGKGLEWIGYI |
| | Antibody 11 | YYSGSTNYNPSLKSRVTISVDTSKNQFSLKLSSVTAADTAVYYCVSLVYCG |
| | | GDCYSGFDYWGQGTLVTVSS |
| 136 | V_{H} of Comparative | EVQLQQSGPELVKPGASVKMSCKASGYTFTSYFIHWVKQKPGQGLEWIGFI |
| | Antibody 12 | NPYNDGSKYNEKFKGKATLTSDKSSSTAYMELSSLTSEDSAVYYCTRDDG |
| | | YYGYAMDYWGQGTSVTVSS |
| 137 | V_{H} CDR1 of Comparative | SYYWS |
| | Antibody 11 | |
| 138 | V_{H} CDR1 of Comparative | SYFIH |
| | Antibody 12 | |
| 139 | V_{H} CDR2 of Comparative | YIYYSGSTNYNPSLKS |
| | Antibody 11 | |
| 140 | V_{H} CDR2 of Comparative | FINPYNDGSKYNEKFKG |
| | Antibody 12 | |
| 141 | V_{H} CDR3 of Comparative | LVYCGGDCYSGFDY |
| | Antibody 11 | |
| 142 | V_{H} CDR3 of Comparative | DDGYYGYAMDY |
| | Antibody 12 | |
| 143 | V_{L} of Comparative | DIQLTQSPSSLSASVGDRVSFTCQASQDINNFLNWYQQKPGKAPKLLIYDA |
| | Antibody 11 | SNLETGVPSRFSGSGSGTDFTFTISSLQPEDIATYYCQQYGNLPFTFGGGT |
| | | KVEIKR |
| 144 | V_{L} of Comparative | DIQMTQSPSSLSASLGERVSLTCRATQELSGYLSWLQQKPDGTIKRLIYAAS |
| | Antibody 12 | TLDSGVPKRFSGNRSGSDYSLTISSLESEDFADYYCLQYAIYPYTFGGGTKL |
| | | EIKR |
| 145 | V_{L} CDR1 of Comparative | QASQDINNFLN |
| | Antibody 11 | |
| 146 | V_{L} CDR1 of Comparative | RATQELSGYLS |
| | Antibody 12 | |
| 147 | V_{L} CDR2 of Comparative | DASNLET |
| | Antibody 11 | |
| 148 | V_{L} CDR2 of Comparative | AASTLDS |
| | Antibody 12 | |
| 149 | V_{L} CDR3 of Comparative | QQYGNLPFT |
| | Antibody 11 | |
| 150 | V_{L} CDR3 of Comparative | LQYAIYPYT |
| | Antibody 12 | |

1. An antibody, or antigen binding fragment, which binds to CLL1 selected from the group consisting of:
Antibody 5, comprising:
(a) a heavy chain variable region comprising,
(i) a CDR1 of SEQ ID NO:27,
(ii) a CDR2 of SEQ ID NO:37, and
(iii) a CDR3 of SEQ ID NO:47; and
(b) a light chain variable region comprising,
(i) a CDR1 of SEQ ID NO:67,
(ii) a CDR2 of SEQ ID NO:77, and
(iii) a CDR3 of SEQ ID NO:87;
and,
Antibody 1, comprising:
(a) a heavy chain variable region comprising,
(i) a CDR1 of SEQ ID NO:23,
(ii) a CDR2 of SEQ ID NO:33, and
(iii) a CDR3 of SEQ ID NO:43; and
(b) a light chain variable region comprising,
(i) a CDR1 of SEQ ID NO:63,
(ii) a CDR2 of SEQ ID NO:73, and
(iii) a CDR3 of SEQ ID NO:83;
and,
Antibody 2, comprising:
(a) a heavy chain variable region comprising,
(i) a CDR1 of SEQ ID NO:24,
(ii) a CDR2 of SEQ ID NO:34, and
(iii) a CDR3 of SEQ ID NO:44; and
(b) a light chain variable region comprising,
(i) a CDR1 of SEQ ID NO:64,
(ii) a CDR2 of SEQ ID NO:74, and
(iii) a CDR3 of SEQ ID NO:84;
and,
Antibody 3, comprising;
(a) a heavy chain variable region comprising,
(i) a CDR1 of SEQ ID NO:25,
(ii) a CDR2 of SEQ ID NO:35, and
(iii) a CDR3 of SEQ ID NO:45; and
(b) a light chain variable region comprising,
(i) a CDR1 of SEQ ID NO:65,
(ii) a CDR2 of SEQ ID NO:75, and
(iii) a CDR3 of SEQ ID NO:85;
and,
Antibody 4, comprising:
(a) a heavy chain variable region comprising,
(i) a CDR1 of SEQ ID NO:26,
(ii) a CDR2 of SEQ ID NO:36, and
(iii) a CDR3 of SEQ ID NO:46; and
(b) a light chain variable region comprising,
(i) a CDR1 of SEQ ID NO:66,
(ii) a CDR2 of SEQ ID NO:76, and
(iii) a CDR3 of SEQ ID NO:86;
and,
Antibody 6, comprising:
(a) a heavy chain variable region comprising,
(i) a CDR1 of SEQ ID NO:28,
(ii) a CDR2 of SEQ ID NO:38, and
(iii) a CDR3 of SEQ ID NO:48; and
(b) a light chain variable region comprising,
(i) a CDR1 of SEQ ID NO:68,
(ii) a CDR2 of SEQ ID NO:78, and
(iii) a CDR3 of SEQ ID NO:88;
and,
Antibody 7, comprising:
(a) a heavy chain variable region comprising,
(i) a CDR1 of SEQ ID NO:29,
(ii) a CDR2 of SEQ ID NO:39, and
(iii) a CDR3 of SEQ ID NO:49; and
(b) a light chain variable region comprising,
(i) a CDR1 of SEQ ID NO:69,
(ii) a CDR2 of SEQ ID NO:79, and
(iii) a CDR3 of SEQ ID NO:89;
and,
Antibody 8, comprising:
(a) a heavy chain variable region comprising,
(i) a CDR1 of SEQ ID NO:30,
(ii) a CDR2 of SEQ ID NO:40, and
(iii) a CDR3 of SEQ ID NO:50; and
(b) a light chain variable region comprising,
(i) a CDR1 of SEQ ID NO:70,
(ii) a CDR2 of SEQ ID NO:80, and
(iii) a CDR3 of SEQ ID NO:90;
and,
Antibody 9, comprising:
(a) a heavy chain variable region comprising,
(i) a CDR1 of SEQ ID NO:31,
(ii) a CDR2 of SEQ ID NO:41, and
(iii) a CDR3 of SEQ ID NO:51; and
(b) a light chain variable region comprising,
(i) a CDR1 of SEQ ID NO:71,
(ii) a CDR2 of SEQ ID NO:81, and
(iii) a CDR3 of SEQ ID NO:91;
and,
Antibody 10, comprising:
(a) a heavy chain variable region comprising,
(i) a CDR1 of SEQ ID NO:32,
(ii) a CDR2 of SEQ ID NO:42, and
(iii) a CDR3 of SEQ ID NO:52; and
(b) a light chain variable region comprising,
(i) a CDR1 of SEQ ID NO:72,
(ii) a CDR2 of SEQ ID NO:82, and
(iii) a CDR3 of SEQ ID NO:92.

2. The antibody, or antigen binding fragment, of claim 1, is comprised in a single chain variable fragment (scFv).

3. The antibody, or antigen binding fragment, of claim 2, wherein the scFv is selected from the group consisting of: SEQ ID NO. 3; SEQ ID NO. 4; SEQ ID NO. 5; SEQ ID NO. 6; SEQ ID NO. 7; SEQ ID NO. 8, SEQ ID NO. 9; SEQ ID NO. 10; SEQ ID NO. 11; and SEQ ID NO. 12.

4. The antibody, or antigen binding fragment, of any one of claims 1 to 3, wherein the antibody is conjugated to a synthetic molecule.

5. The antibody, or antigen binding fragment, of claim 4, wherein the antibody is a chimeric antigen receptor and the synthetic molecule comprises a transmembrane region and an intracellular T-cell receptor signaling domain.

6. The antibody, or antigen binding fragment, of claim 5, wherein the transmembrane region is from CD8A or CD28 and/or the intracellular T-cell receptor signaling domain is from CD247 (CD3-zeta).

7. The antibody, or antigen binding fragment, of claim 6, further comprising an intracellular domain of a costimulatory protein receptor.

8. The antibody, or antigen binding fragment, of claim 4, wherein the antibody is a bi-specific T-cell engager (BiTE) and the synthetic molecule comprises an antigen binding domain which binds to a T-cell antigen.

9. The antibody, or antigen binding fragment, of claim 8, wherein the antigen binding domain comprises an antibody fragment that specifically binds CD3.

10. The antibody, or antigen binding fragment, of claim 4, wherein the antibody is an antigen-drug conjugate and the synthetic molecules is a cytotoxic drug or a therapeutic radioisotope.

11. A vector encoding the antibody, or antigen binding fragment, of any one of claims 1 to 10.

12. A pharmaceutical composition comprising the antibody or antigen binding fragment, of any one of claims 1 to 10 and a pharmaceutically acceptable carrier.

13. A chimeric antigen receptor comprising:
a) The antibody or antigen binding fragment as claimed in any one of claims 1 to 10;
b) A transmembrane region; and
c) An intracellular signaling domain.

14. The chimeric antigen receptor of claim 13, wherein the transmembrane region is from CD8A or CD28 and/or the intracellular signaling domain is from CD247 (CD3-zeta).

15. The chimeric antigen receptor of claim 13, further comprising an intracellular signaling domain of a costimulatory protein receptor.

16. The chimeric antigen receptor of any one of claims 13 to 15, wherein the CAR comprises:
a. a human CD247 (CD3-zeta) cytoplasmic domain (SEQ ID NO:113), a human CD137 co-stimulatory domain (SEQ ID NO:114); a human CD8A transmembrane domain (SEQ ID NO: 115), a human CD8A hinge domain (SEQ ID NO:116); antibody or antigen binding fragment as claimed in any one of claims 1 to 12; and an CD8A extracellular signal peptide (SEQ ID NO:119); or
b. a human CD247 (CD3-zeta) cytoplasmic domain (SEQ ID NO:113), a human CD28 co-stimulatory domain (SEQ ID NO:120); a human CD28 transmembrane domain (SEQ ID NO: 121), a human CD28 hinge domain (SEQ ID NO:122); antibody or antigen binding fragment as claimed in any one of claims 1 to 12; and an CD8A extracellular signal peptide (SEQ ID NO:119).

17. The chimeric antigen receptor of any one of claims 13 to 16, wherein the CAR has a sequence selected from SEQ ID NO: 93; SEQ ID NO. 94; SEQ ID NO. 95; SEQ ID NO. 96; SEQ ID NO. 97; SEQ ID NO. 98; SEQ ID NO. 99, SEQ ID NO. 100; SEQ ID NO. 101; and SEQ ID NO. 102.

18. A recombinant T cell comprising the chimeric antigen receptor of any one of claims 13 to 17.

19. A bispecific T-cell engager comprising:
a) The antibody or antigen binding fragment as claimed in any one of claims 1 to 10;
b) An antigen binding domain which binds to a T-cell antigen; and
c) A linker joining (a) and (b).

20. The bi-specific T-cell engager of claim 19, wherein the antigen binding domain comprises an antigen binding fragment that specifically binds CD3.

21. The bi-specific T-cell engager of claim 20, wherein the antibody fragment that specifically binds CD3 comprises:
(a) a heavy chain variable region comprising,
(i) a CDR1 of SEQ ID NO:123,
(ii) a CDR2 of SEQ ID NO:124, and
(iii) a CDR3 of SEQ ID NO:125; and
(b) a light chain variable region comprising,
(i) a CDR1 of SEQ ID NO:126,
(ii) a CDR2 of SEQ ID NO:127, and
(iii) a CDR3 of SEQ ID NO:128;

22. An antibody-drug conjugate comprising:
a) The antibody or antigen binding fragment as claimed in any one of claims 1 to 10;
b) A therapeutic drug substance;
c) A linker joining (a) and (b).

23. The antibody-drug conjugate of claim 22, wherein the synthetic molecule is a cytotoxic drug.

24. An antibody, or antigen binding fragment, of any one of claims 1 to 10, a pharmaceutical composition of claim 12, a chimeric antigen receptor of any one of claims 13 to 17, a recombinant T-cell of claim 18, a bi-specific T-cell engager of any one of claims 19 to 21 or the antibody-drug conjugate of any one of claims 22 to 23 for use as a medicament.

25. An antibody, or antigen binding fragment, of any one of claims 1 to 10, a pharmaceutical composition of claim 12, a chimeric antigen receptor of any one of claims 13 to 17, a recombinant T-cell of claim 18, a bi-specific T-cell engager of any one of claims 19 to 21 or the antibody-drug conjugate of any one of claims 22 to 23 for use in killing or inhibiting the growth of cells expressing CLL1.

26. An antibody, or antigen binding fragment, of any one of claims 1 to 10, a pharmaceutical composition of claim 12, a chimeric antigen receptor of any one of claims 13 to 17, a recombinant T-cell of claim 18, a bi-specific T-cell engager of any one of claims 19 to 21 or the antibody-drug conjugate of any one of claims 22 to 23 for use in medicine.

27. An antibody, or antigen binding fragment, of any one of claims 1 to 10, a pharmaceutical composition of claim 12, a chimeric antigen receptor of any one of claims 13 to 17, a recombinant T-cell of claim 18, a bi-specific T-cell engager of any one of claims 19 to 21 or the antibody-drug conjugate of any one of claims 22 to 23 for use in the treatment of a disease or disorder wherein the disease or disorder is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

28. A method of treating a disease or condition associated with the expression of CLL1, said method comprising administering to a patient in need thereof an effective amount of an antibody, or antigen binding fragment, of any one of claims 1 to 10, a pharmaceutical composition of claim 12, a chimeric antigen receptor of any one of claims 13 to 17, a recombinant T-cell of claim 18, a bi-specific T-cell engager of any one of claims 19 to 21 or the antibody-drug conjugate of any one of claims 22 to 23 to a patient in need thereof.

29. The method of treatment of claim 28, wherein the disease or condition is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.
